(19) United States
(12) Patent Application Publication
(10) Pub. No.: US 2021/0134179 A1
(43) Pub. Date: May 6, 2021

(54) Systems And Methods For Managing Dynamic User Interactions With Online Services For Enhancing Mental Health Of Users

(71) Applicant: Happify, Inc., New York, NY (US)

(72) Inventors: Ran ZILCA, Ra'anana (IL); Tomer BEN-KIKI, New York, NY (US); Derrick CARPENTER, Middletown, CT (US)

(21) Appl. No.: 17/060,831

(22) Filed: Oct. 1, 2020

Related U.S. Application Data
(60) Provisional application No. 62/935,126, filed on Nov. 14, 2019.
(60) Provisional application No. 62/928,023, filed on Oct. 30, 2019.

(51) Int. Cl. G09B 19/00; G16H 20/70; G16H 10/20; G16H 40/20; G16H 70/20; G06F 16/9535; G06F 40/40

(52) U.S. Cl. CPC G09B 19/00; G16H 20/70; G16H 10/20; G06F 40/40; G16H 70/20; G06F 16/9535; G16H 40/20

(57) ABSTRACT

A system for conducting dialogues with users of an online service recommending N activities comprises a processor to generate a first file including M portions for conducting the dialogues. The processor generates N second files for the N activities, respectively. The processor includes in each of the N second files references to a plurality of the M portions of the first file. The processor generates a plurality of third files, each corresponding to a task for performing one of the N activities. The processor conducts a dialogue with one of the users about one of the N activities using one of the N second files corresponding to the one of the N activities, a plurality of the M portions of the first file referenced by the one of the N second files, and one of the third files corresponding to a task for performing the one of the N activities.

### CROSS-REFERENCE TO RELATED APPLICATIONS

This application claims the benefit of U.S. Provisional Application No. 62/928,023, filed on Oct. 30, 2019 and U.S. Provisional Application No. 62/935,126, filed on Nov. 14, 2019. The entire disclosures of the applications referenced above are incorporated herein by reference.

### FIELD

The present disclosure relates generally to online services for enhancing mental health of users and more particularly to systems and methods for managing dynamic user interactions with the online services for enhancing mental health of users.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Digital therapeutics, a subset of digital health, are evidence-based therapeutic interventions, driven by software programs to prevent, manage, or treat a metal disorder or disease. The treatment relies on behavioral and lifestyle changes usually spurred by a collection of digital impetuses. Due to the digital nature of the methodology, data can be collected and analyzed as both a progress report and a preventative measure. Although digital therapeutics can be employed in many ways, the term can be broadly defined as a treatment or therapy that utilizes digital and often Internet-based health technologies to spur changes in patient behavior. Digital therapeutics differ from wellness apps or medication reminder apps in that digital therapeutics require rigorous clinical evidence to substantiate intended use and impact on disease state.

### SUMMARY

A system for conducting dialogues with users of an online service recommending N activities for enhancing mental health of the users, where N is an integer greater than 1, comprises a processor and memory storing instructions. The instructions, when executed by the processor, configure the processor to receive, at the system, an input from a user via a device of the user to initiate a dialogue with the online service about an activity recommended to the user by the online service from the N activities. The instructions, when executed by the processor, configure the processor to identify a first file in the system corresponding to the activity from N files based on the input. The N files respectively correspond to the N activities. The instructions, when executed by the processor, configure the processor to include references in the first file to a plurality of portions of a second file in the system to conduct the dialogue. The second file includes M portions for conducting dialogues about the N activities, where M is less than N. The plurality of portions are selected from the M portions based on the activity. The instructions, when executed by the processor, configure the processor to identify a third file in the system corresponding to a task for performing the activity. The third file represents data for presenting to the user in the dialogue about the activity. The instructions, when executed by the processor, configure the processor to compile, at the system, the first file, the plurality of portions of the second file, and the third file to generate a handler to handle the dialogue about the activity. The instructions, when executed by the processor, configure the processor to receive, at the system, additional inputs from the user via the device of the user. The instructions, when executed by the processor, configure the processor to conduct the dialogue with the user on the device of the user based on the additional inputs using the handler to further enhance mental health of the user.

In other features, the instructions further configure the processor to conduct any number of dialogues with any number of users about any of the N activities using the N files, the second file, and at least N of the third file. Each of the N third files corresponds to a task for performing the N activities, respectively.

In other features, the instructions further configure the processor to reuse at least one of the plurality of portions of the second file to conduct a second dialogue about a second one of the N activities with a second user of the online service.

In other features, the instructions further configure the processor to reuse a plurality of the M portions of the second file to conduct more than one dialogue about more than one of the N activities with more than one user of the online service.

In other features, the instructions further configure the processor to include a variable with a generic value in one of the plurality of portions of the second file, and to allow the first file to assign a specific value from the third file to the variable.

In other features, the instructions further configure the processor to include a variable with a first value in one of the plurality of portions of the second file, and to allow the first file to overwrite the first value with a second value from the third file.

In other features, the instructions further configure the processor to include a variable with a default value in one of the plurality of portions of the second file, and to allow the default value to persist in the dialogue by entering a null value for the variable in the third file.

In other features, the instructions further configure the processor to conduct the dialogue based on a flow of the plurality of portions of the second file, and to control the flow in an order that is different than that in which the plurality of portions are arranged in the second file.

In still other features, a system for conducting dialogues with users of an online service recommending N activities for enhancing mental health of the users, where N is an integer greater than 1, comprises a processor and memory storing instructions. The instructions, when executed by the processor, configure the processor to generate a first master file including M portions for conducting the dialogues with the users of the online service about the N activities, where M is less than N. The instructions, when executed by the processor, configure the processor to generate N second files for the N activities, respectively. The instructions, when executed by the processor, configure the processor to include in each of the N second files references to a plurality of the M portions of the first file. The instructions, when executed by the processor, configure the processor to generate a plurality of third files. Each of the third files corresponds to a task for performing one of the N activities. The instructions, when executed by the processor, configure the processor to conduct a dialogue with one of the users of the online service about one of the N activities using one of the N second files corresponding to the one of the N activities, a plurality of the M portions of the first file referenced by the one of the N second files, and one of the third files corresponding to a task for performing the one of the N activities. The one of the N activities is associated with mental health of the user. The dialogue enhances the mental health of the user.

In other features, the instructions further configure the processor to conduct any of the dialogues with any of the users about any of the N activities using the first file, the N second files, and the plurality of third files.

In other features, the instructions further configure the processor to reuse at least one of the plurality of the M portions of the first file to conduct a second dialogue about a second one of the N activities with a second one of the users of the online service.

In other features, the instructions further configure the processor to reuse one or more of the M portions of the first file to conduct more than one dialogue about more than one of the N activities with more than one user of the online service.

In other features, the instructions further configure the processor to compile, the one of the N second files corresponding to the one of the N activities, the plurality of the M portions of the first file referenced by the one of the N second files, and the one of the third files corresponding to the task for performing the one of the N activities, to generate a handler. The instructions further configure the processor to conduct the dialogue using the handler.

In other features, the instructions further configure the processor to include variables with assignable values in some of the M portions of the first file, to include data in the third files for presenting to the users in the dialogues, and to allow some of the N second files to assign a portion of the data from the third files to a portion of the assignable values of the variables when conducting the dialogues.

In other features, the instructions further configure the processor to include variables with generic values in the M portions of the first file, to include data in the third files for presenting to the users in the dialogues, and to allow some of the N second files to assign specific values from a portion of the data in the third files to a portion of the variables when conducting the dialogues.

In other features, the instructions further configure the processor to include a variable with a first value in one of the M portions of the first file, to include data in the third files for presenting to the users in the dialogues, and to allow one of the N second files to overwrite the first value with a second value from one of the third files.

In other features, the instructions further configure the processor to include a variable with a default value in one of the M portions of the first file, to include data in the third files for presenting to the users in the dialogues, and to allow the default value to persist in one of the dialogues by entering a null value for the variable in one of the third files.

In other features, the instructions further configure the processor to conduct the dialogue based on a flow of the plurality of the M portions of the first file, and to control the flow in an order that is different than that in which the plurality of the M portions of the first file are arranged in the first file.

In other features, the instructions further configure the processor to receive an input from the one of the users via a device of the one of the users to initiate the dialogue, to identify the one of the N second files based on the input, to receive additional inputs from the user via the device of the user, and to conduct the dialogue on the device of the user based on the additional inputs.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows an example of a client-server based distributed communication system that can be used to implement an online service for enhancing mental health of users and a dialogue management system for the online service;

FIG. 2 shows an example of a client device of the distributed communication system of FIG. 1;

FIG. 3 shows an example of a block diagram of a server of the distributed communication system of FIG. 1;

FIG. 4 shows an example of a block diagram of the online service;

FIG. 5A shows an example of a block diagram of the dialogue management system;

FIG. 5B shows an example of a dialogue box (also called a dialog box) including a dialogue between the online service of FIG. 4 and a user of the online service using the dialogue management system of FIG. 5A;

FIG. 6 shows an example of a flowchart of a method of conducting a dialogue between the online service of FIG. 4 and a user of the online service using the dialogue management system of FIG. 5A;

FIG. 7 shows an example of a flowchart of a method of creating a master dialogue file for conducting a dialogue between the online service of FIG. 4 and a user of the online service using the dialogue management system of FIG. 5A;

FIG. 8 shows an example of a flowchart of a method of creating a skeleton file for conducting a dialogue between the online service of FIG. 4 and a user of the online service using the dialogue management system of FIG. 5A;

FIG. 9 shows an example of a flowchart of a method of creating a skin file for conducting a dialogue between the online service of FIG. 4 and a user of the online service using the dialogue management system of FIG. 5A;

FIGS. 10A-10N show a table including examples of tracks and activities offered by the online service to the users of the online service of FIG. 4 for improving mental health of the users; and

FIGS. 11A-11C show a table including an example of a track, activities of the track, and tasks of the activities offered by the online service for FIG. 4 for improving mental health of the users.

In the drawings, reference numbers may be reused to identify similar and/or identical elements.

### DETAILED DESCRIPTION

The present disclosure relates to a tiered architecture of a dialogue management system used to present dialogue boxes (also called dialog boxes) to users to interact with an online system used for providing, among other things, activities and tasks to enhance mental health of the users. The dialogue boxes are presented to the users on their respective computing devices such as smartphones, tablets, laptops, etc. The users can interact with the online system by sharing an experience about an activity, for example, via the dialogue boxes. The users can share/discuss their experience from performing an activity prescribed to them by the online service. The discussion or dialogue with the online service, conducted and managed by the disclosed dialogue management system in a conversational format, helps improve the mental health of the users.

The disclosed dialogue management system provides an efficient and versatile database-based architecture that provides a compact library of a limited number of hierarchically interlinked data structures in three layers that can conduct dialogues with multiple users on any of a host of activities prescribed by the online service. The library synergistically reuses some of the data structures, which significantly simplifies the design of the dialogue management system and minimizes resources used by the databases of the online service to conduct the dialogues while also providing engaging dialogues with users with a real-world, life-like feeling that otherwise requires complex design and enormous amounts of resources.

For example, a user may wish to discuss an activity performed by the user from a track of activities recommended to the user by the online service to improve an aspect of mental health such as a happiness skill of the user. The user can share his or her experience with the online service by conversing with the online service via a dialogue box (also called a dialog box). The user can type (or speak) an input into the dialogue box indicating the topic of discussion. For example, the topic can be based on the activity performed by the user from a recommended track. The topic can be the user's experience from performing the activity. The dialogue management system, using its tiered architecture, which functions as a conversational agent, provides responses to the user inputs. Together, the user's inputs and the online service's responses occur in the form an interactive discussion about the topic of the user's interest.

More generally, while the dialogue session is interactive, the dialogue management system leads the interaction as an intervention that has an agenda (e.g., improving the user's skill for expressing gratitude) and uses an adherence fidelity module of the online service for that purpose. In the course of a dialogue, sometimes the user takes the lead and the dialogue management system responds, and sometimes the dialogue management system takes the lead and the user responds. Regardless, the interaction is more strategically guided by the dialogue management system such that the user completes a beneficial intervention. Notably, this feature of the dialogue management system is different from interactive agents in other domains like customer service where, the goal of the dialogue is only a user goal. Instead, in the dialogue management system of the present disclosure, both parties in the interaction—the dialogue management system and the user—have goals. In that sense, the dialogue management system conducts the dialogues with the users based on a “mixed initiative”.

As can be appreciated, the user inputs in these dialogues can vary widely from user to user and across activities, and yet the dialogue management system can comprehend the variations in the inputs and can provide highly relevant responses due to the tiered architecture of the dialogue management system. The tiered architecture of the dialogue management system makes possible such seamless interactions with the users regardless of the variety in the activities and tasks and regardless of the variations in the user inputs. The tiered architecture of the dialogue management system is described below in detail.

Essentially, the dialogue management system is a conversational agent that delivers conversational interventions to users, where the conversations may be conducted via text, audio, video, virtual reality (VR), or a combination thereof. The dialogue session can be similar to a text messaging session. The dialogues are designed to steer the user to adhere to the recommended intervention (e.g., following a recommended track). For example, if the intervention is for improving gratitude skill, the dialogue is designed to ensure the user is indeed expressing gratitude; if the intervention is for improving empathy skill, the dialogue is designed to ensure the user is in fact practicing empathy; and so on. The dialogue is designed to provide any corrections needed to maintain the user's adherence.

As explained below in detail, the online service provides more than one task to complete an activity. One task is called You Decide How (YDH) task, where, as the name suggests, the user can decide how to perform the activity. The other tasks may include other contextualized ways to perform the same or similar activity (e.g., expressing gratitude at work or expressing gratitude at home). Ordinarily, system designers would have to script each of these dialogues individually not only for each task per activity and for multiple activities but also by taking into account different conversational styles of different users. As can be appreciated, scripting all possible scenarios of the dialogues can be a laborious and daunting process.

Instead, the disclosed dialogue management system employs a novel master, skeleton, and skin (MSS) framework, where a single master file contains templates of sections or portions of possible dialogues (also called sub-dialogues). For example, the templates could be for a greeting, an ending, trying to get the user to adhere to one or more items, and so on. The templates can be general or generic. The next level is a skeleton, which specifies that a particular activity for which the dialogue is to be conducted comprises a sequence of selected sections or dialogue portions from the master file (e.g., sections or dialogue portions 3, 7, 12, 8, 5, and 17). This is followed by a base skin, which includes all the prompts for conducting the dialogue, which is followed by tasks for the particular activity. These MSS elements for the particular activity for which the dialogue is to be conducted are compiled into a single handler that conducts the dialogue. The process is repeated for each dialogue session.

Thus, the skeleton level includes only those sub-dialogues from the master that are relevant for the particular activity under discussion, and the skins provide the specific inputs that replace the generic values in the selected sub-dialogues from the master to conduct a meaningful dialogue for the particular activity. This way, the dialogue progresses almost as naturally as if occurring between the user and a human, and the user receives responses from the dialogue management system that are consistent with the user's expectations and/or that are designed to steer the user in a direction for effective intervention.

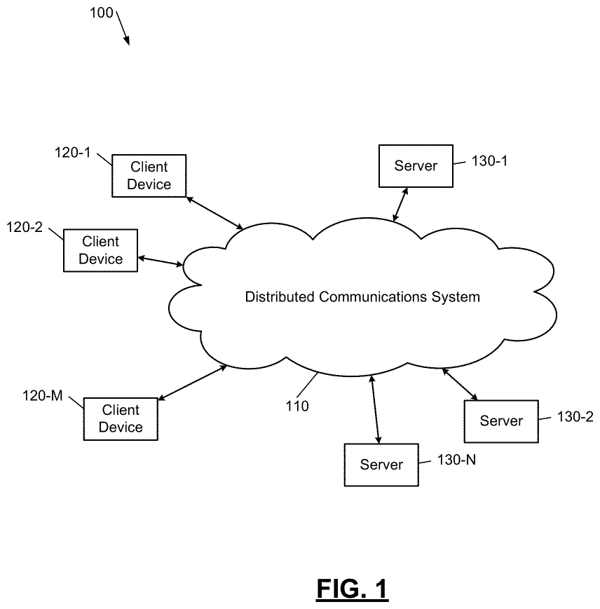

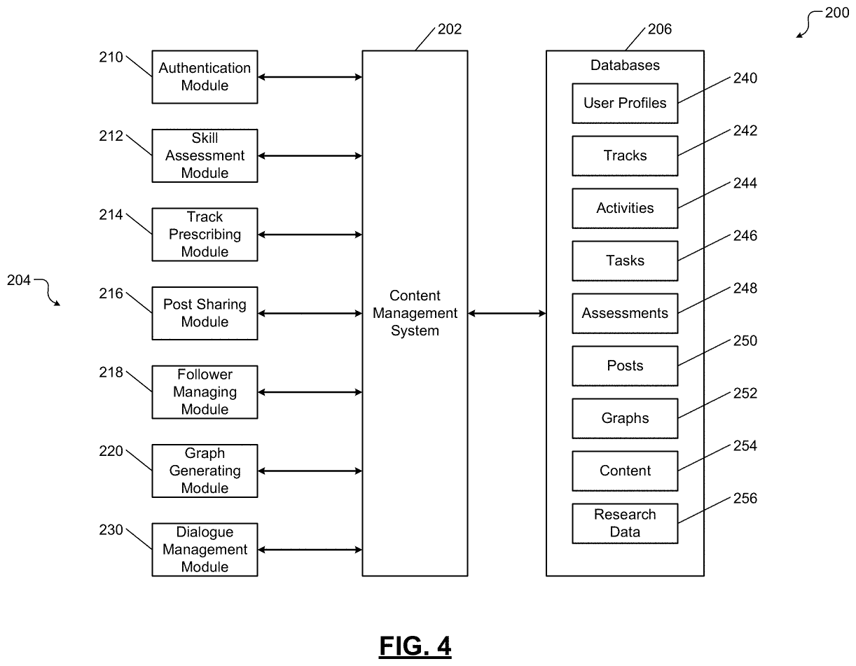

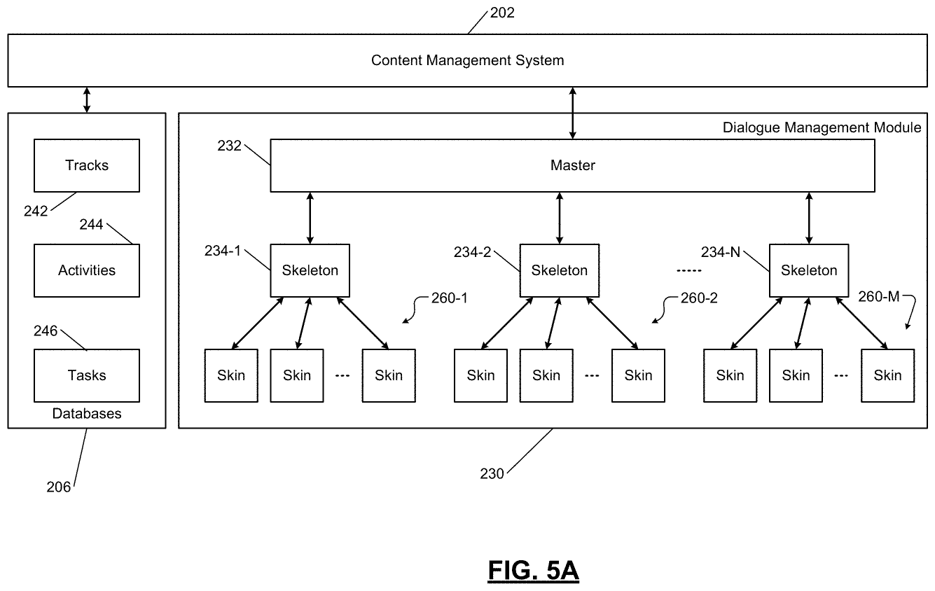

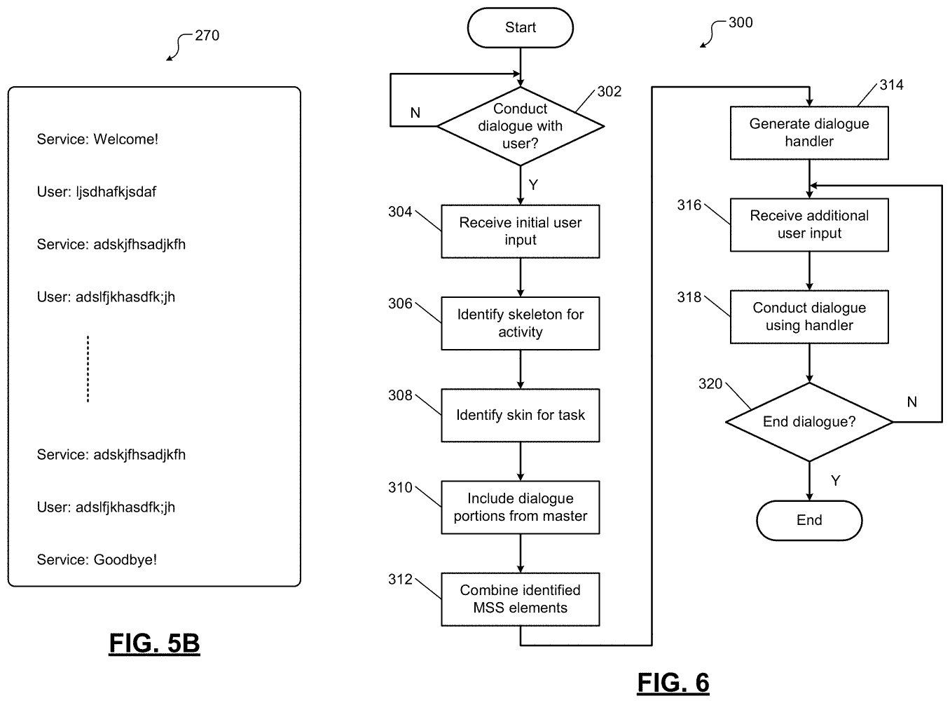

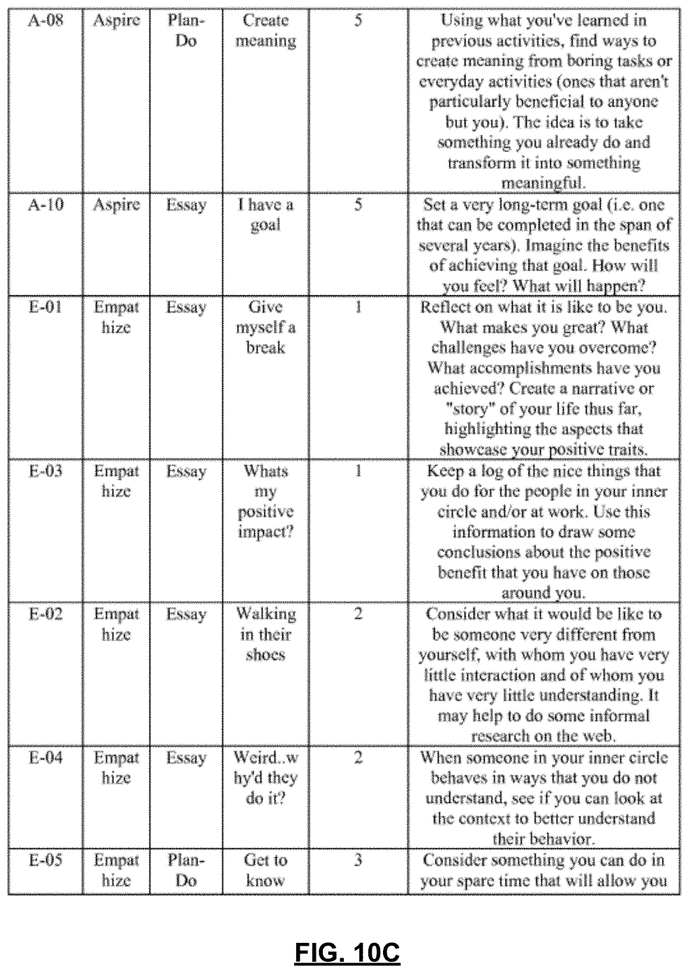

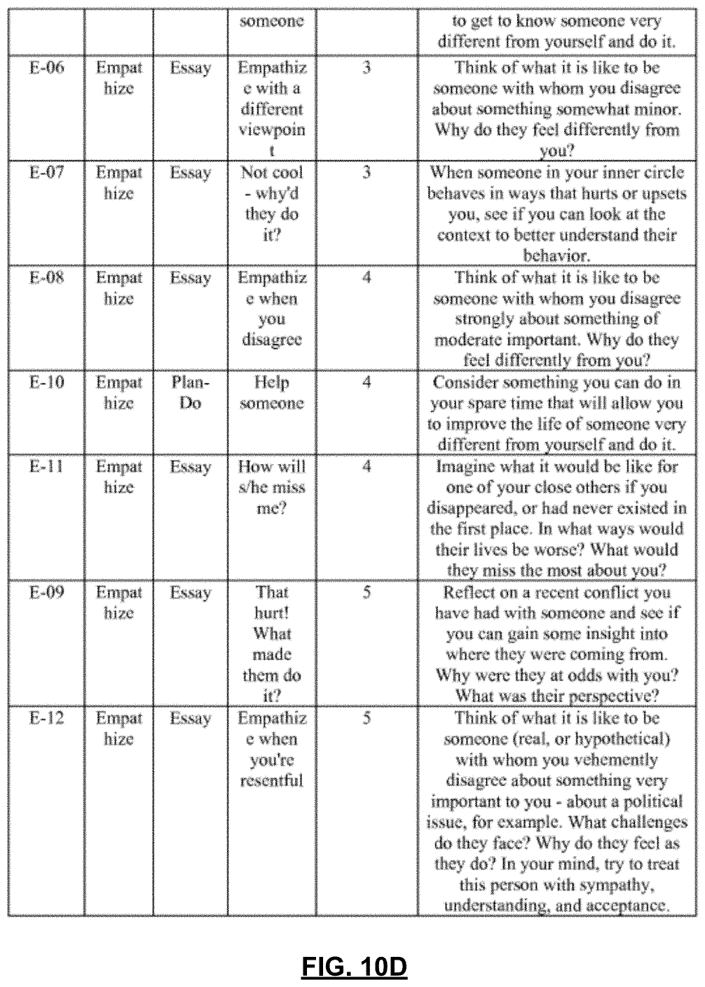

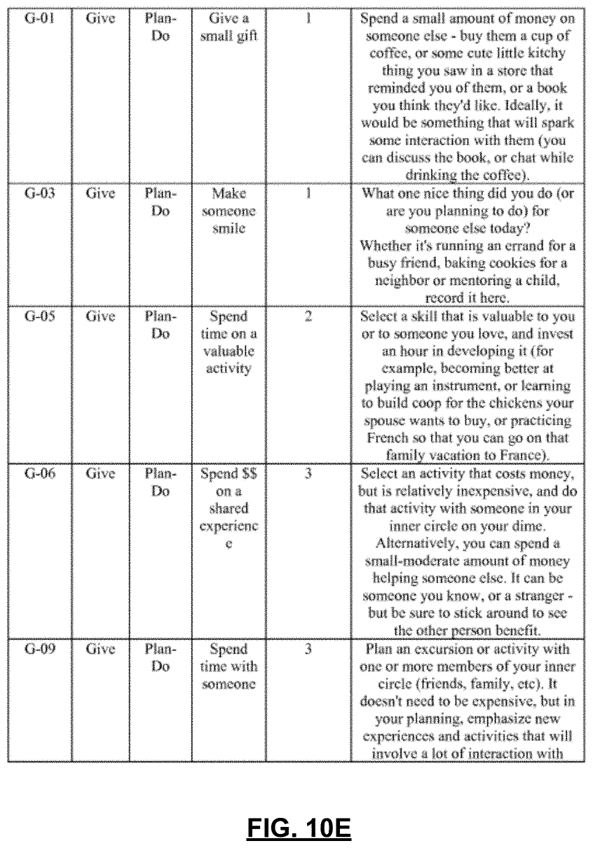

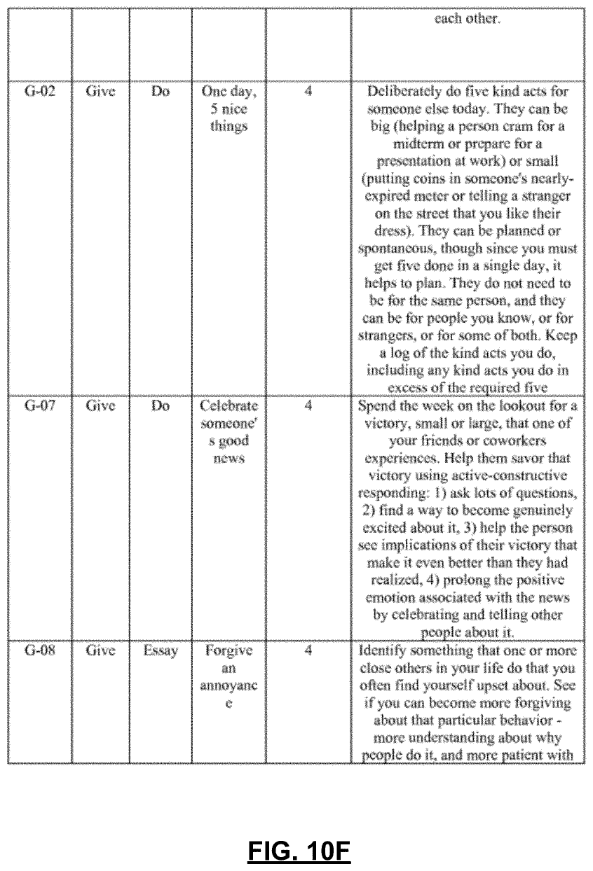

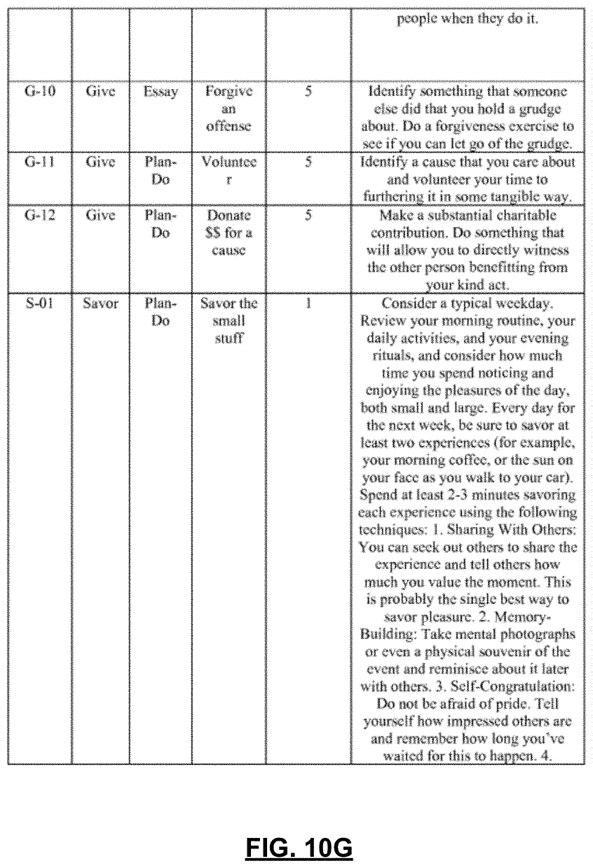

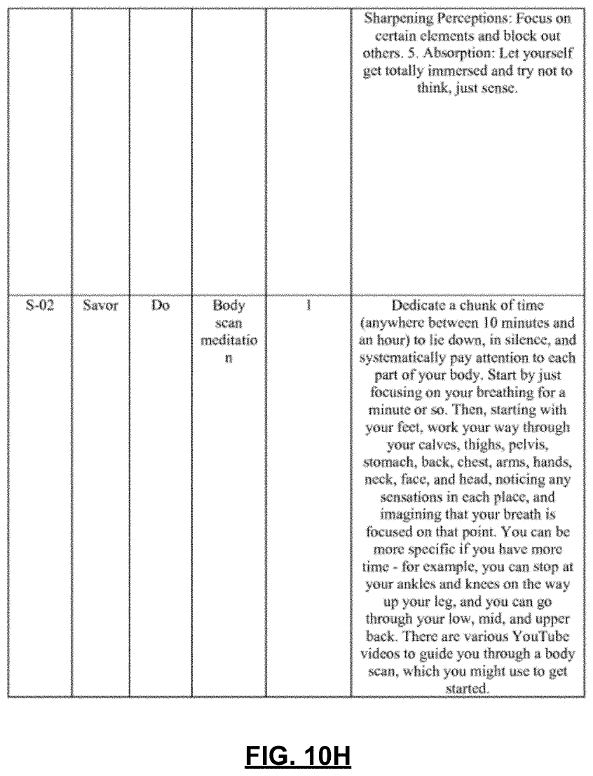

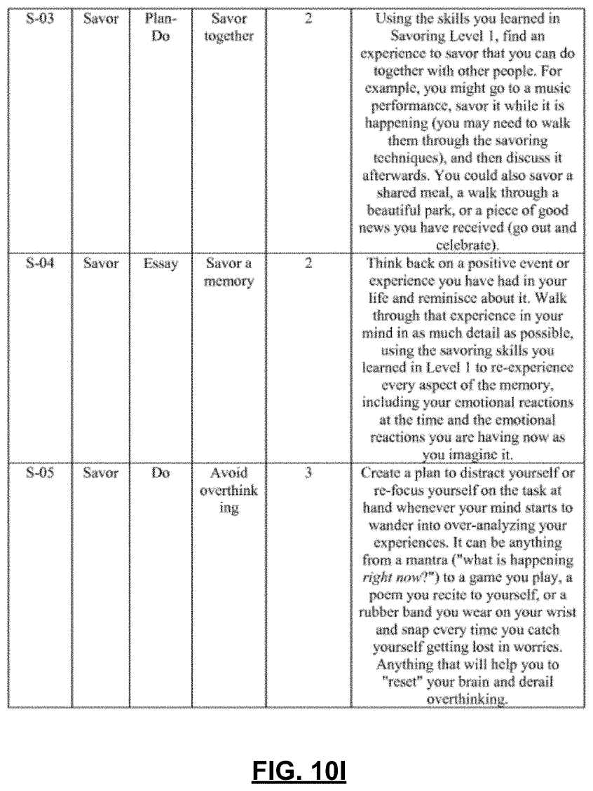

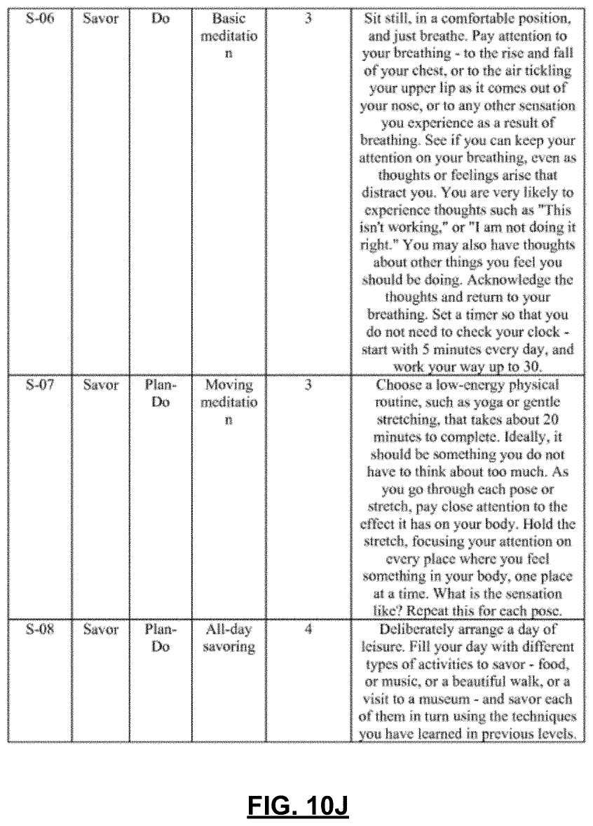

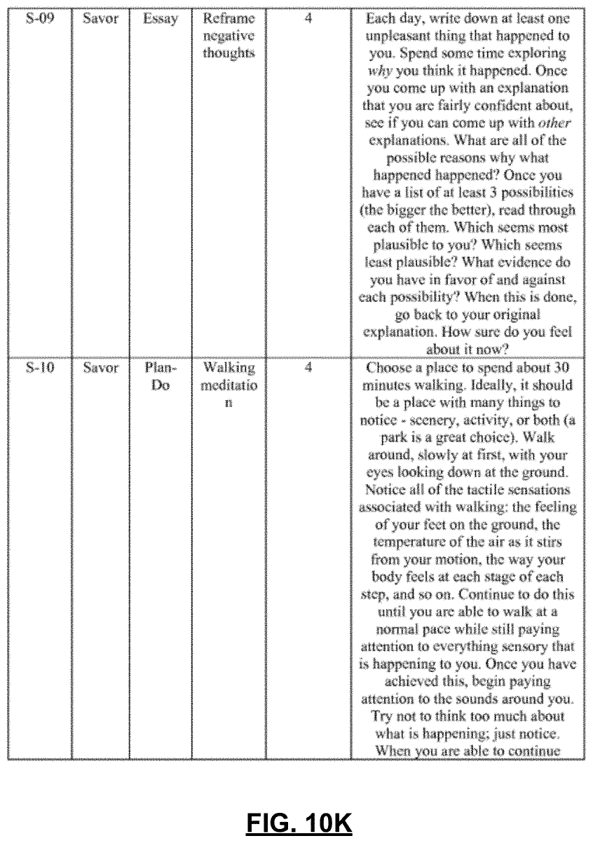

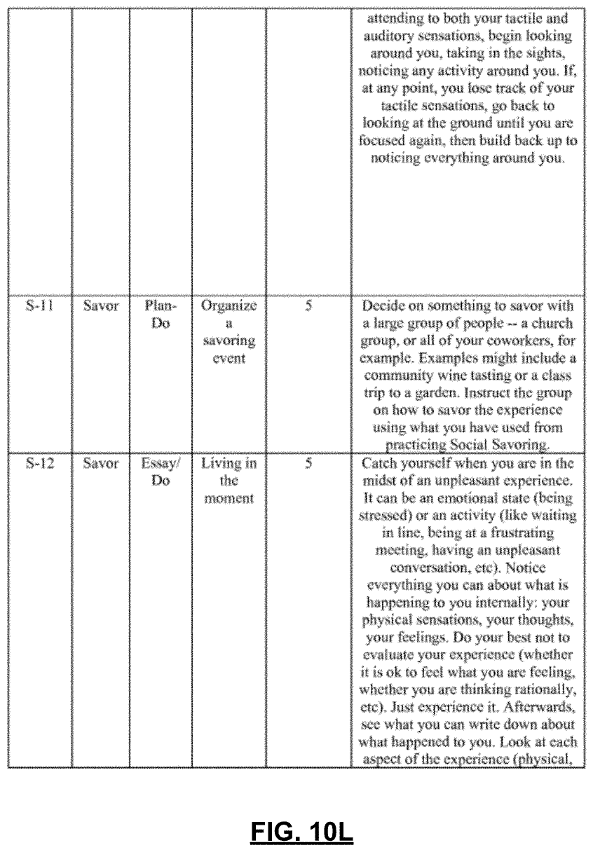

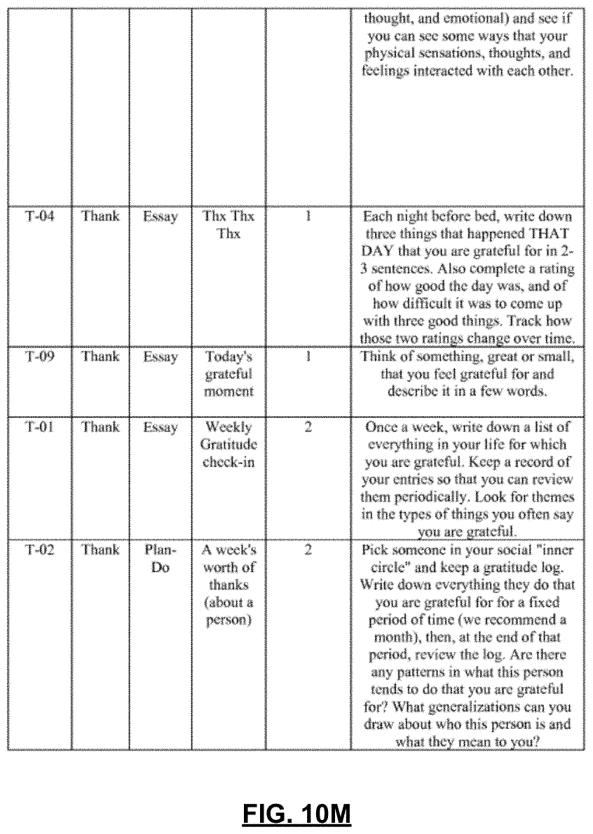

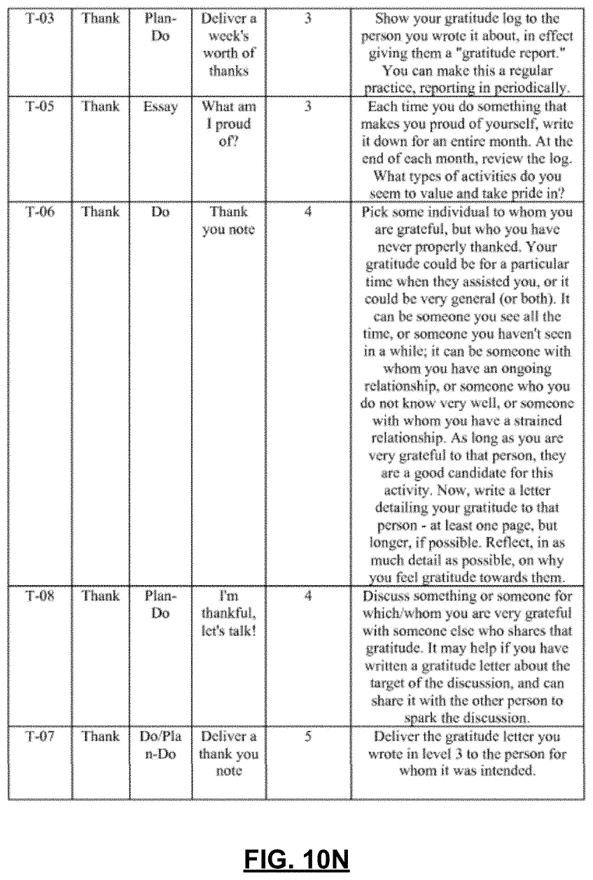

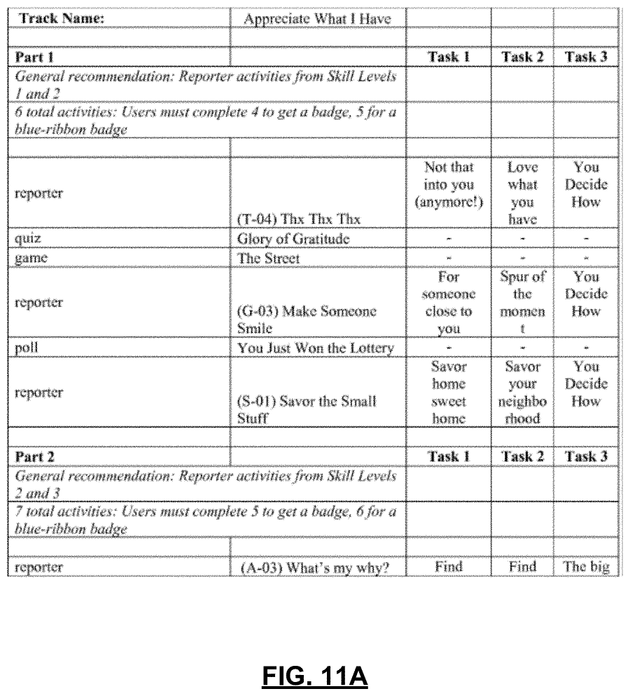

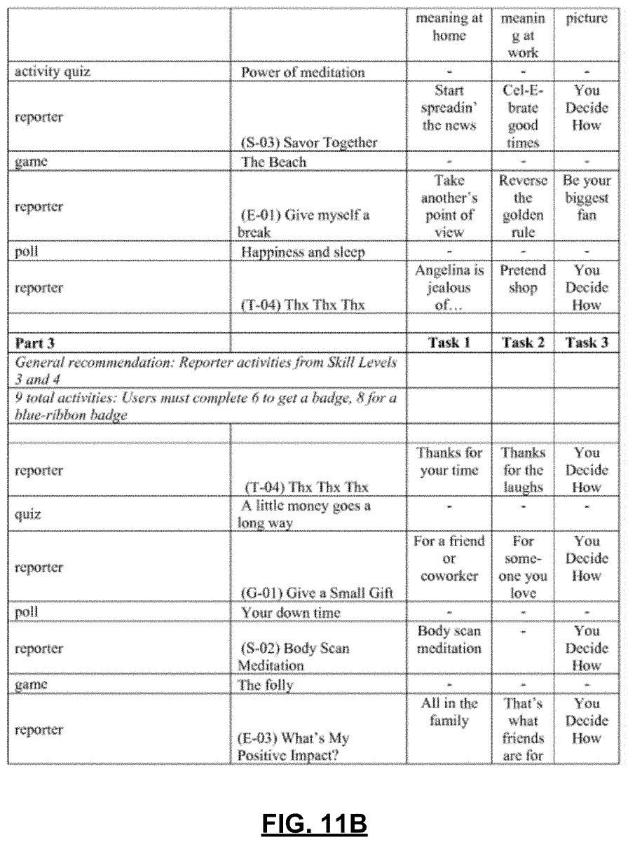

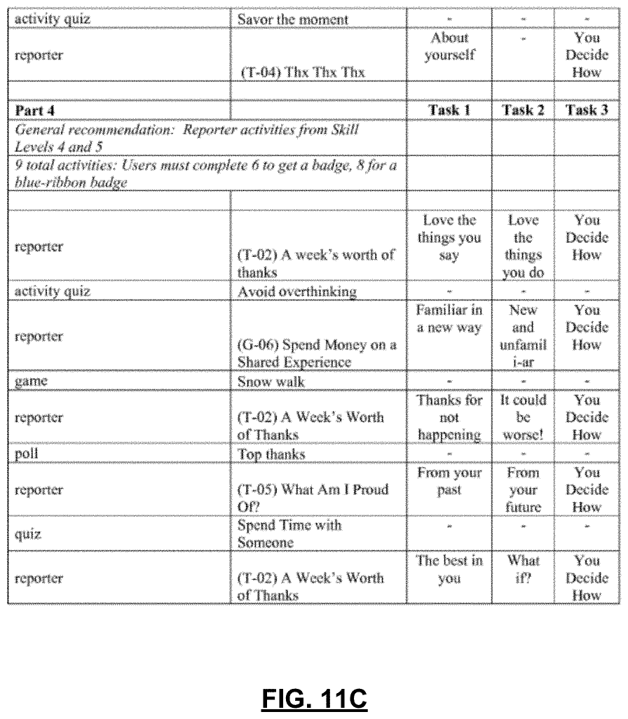

The present disclosure is organized as follows. An example of a client-server based distributed communication system that can implement the online service and the dialogue management system of the present disclosure is shown and described with reference to FIGS. 1-3. Subsequently, to enhance understanding and comprehension of the scope and context of the disclosed dialogue management system, the online service, including the various tracks, activities, and tasks is initially described in detail with reference to FIG. 4. Thereafter, the dialogue management system of the present disclosure is described in detail with reference to FIGS. 5A-9. FIGS. 10A-11C show tables of tracks, activities, and tasks offered by the online service.

Throughout the present disclosure, happiness skills are used only as one example of various aspects of overall mental health. The teachings of the present disclosure apply equally to other aspects of mental health. For example, the online service, including the various tracks, activities, and tasks described with reference to FIG. 4, is described with reference to happiness skills for example only. The scope of the online service and the dialogue management system of the present disclosure is not limited to enhancing happiness skills only. Rather, these systems benefit from, leverage, and utilize vast amounts of clinical data and knowledge obtained therefrom through scientific research.

In many ways, the ultimate goal of mental health is for a person to have happiness, which has several definitions in the scientific literature, all pointing to psychological ingredients. For example, the PERMA model, developed by Martin Seligman, one of the founders of positive psychology, include the following five core elements of psychological well-being and happiness: Positive emotion, Engagement, Relationships, Meaning, and Accomplishment. These are arguably the essential ingredients of happiness. For more information on the PERMA model, see https://positivepsychology.com/perma-model. The online service and the dialogue management system of the present disclosure interactively guide people in developing and mastering these elements to reach the ultimate goal of metal health. A brief overview of each of these elements follows.

Positive emotion is arguably the most obvious connection to happiness. Focusing on positive emotions is the ability to remain optimistic and view one's past, present, and future from a constructive perspective. A positive view can help in relationships and work, and can inspire others to be more creative and take more chances. Positive emotion can help people enjoys the daily tasks in their lives and persevere with challenges they will face by remaining optimistic about eventual outcomes.

Activities that meet the need for engagement flood the body with positive neurotransmitters and hormones that elevate one's sense of well-being. This engagement helps people remain present, as well as synthesize the activities where they find calm, focus, and joy. When time flies during an activity, it is likely because the people involved are experiencing this sense of engagement.

Relationships and social connections are crucial to living meaningful lives. Humans are social animals who are hard-wired to bond and depend on other humans, and hence the basic need for healthy relationships. People thrive on connections that promote love, intimacy, and a strong emotional and physical interaction with other humans. Positive relationships with parents, siblings, peers, coworkers, and friends is a key ingredient to overall joy. Strong relationships also provide support in difficult times that require resilience.

Knowing “why are we on this earth?” is a key ingredient that can drive people towards fulfillment. Religion and spirituality provide many people with meaning, as can working for a good company, raising children, volunteering for a greater cause, and expressing ourselves creatively.

Having goals and ambition in life can help people to achieve things that can give them a sense of accomplishment. One should make realistic goals that can be met. Simply putting in the effort to achieving the goals can provide a sense of satisfaction. When the goals are achieved, a sense of pride and fulfillment can be experienced. Having accomplishments in life is important to push people to thrive and flourish.

For further information, see Seligman M. (2018). PERMA and the building blocks of well-being. The Journal of Positive Psychology, 13(4), 333-335. For yet another model, see Ryff, C. D., & Keyes, C. L. M. (1995). The structure of psychological well-being revisited. Journal of personality and social psychology, 69(4), 719. The online service and the dialogue management system of the present disclosure provide many evidence-based and research-supported interactive tools using which people can develop and master each of these elements and reach the ultimate goal of metal health.

In general, mental health includes emotional, psychological, and social well-being. Mental health affects how people think, feel, and act. Mental health also helps determine how people handle stress, relate to others, and make choices. Mental health is important at every stage of life, from childhood and adolescence through adulthood and old age. Many factors contribute to mental health problems, including biological factors such as genes or brain chemistry, life experiences such as trauma or abuse, family history of mental health problems, and so on. The online service and the dialogue management system of the present disclosure can analyze these factors.

Various feelings or behaviors can be an early warning sign of a problem. For example, the feelings or behaviors can include eating or sleeping too much or too little; pulling away (withdrawal) from people and usual activities; having low or no energy; feeling numb or like nothing matters; having unexplained aches and pain, feeling helpless or hopeless; smoking, drinking, or using drugs more than usual; feeling unusually confused, forgetful, on edge, angry, upset, worried, or scared; yelling or fighting with family and friends; experiencing severe mood swings that cause problems in relationships; having persistent thoughts and memories that can't be expelled from head; hearing voices or believing things that are not true; thinking of harming oneself or others; inability to perform daily tasks like taking care of household things or getting to work or school; and so on. The online service and the dialogue management system of the present disclosure can detect these feelings or behaviors and make recommendations (e.g., therapeutic interventions) to prevent, treat, and/or cure mental health problems.

Positive mental health allows people to realize their full potential, cope with the stresses of life, work productively, make meaningful contributions to their communities, and so on. Ways to maintain positive mental health include getting professional help if needed, connecting with others, staying positive, getting physically active, helping others, getting enough sleep, developing coping skills, and so on. The online service and the dialogue management system of the present disclosure can promote positive mental health among people and help them maintain it by providing scientifically proven techniques such as those described below.

Below are simplistic examples of a distributed computing environment in which the systems and methods of the present disclosure can be implemented. Throughout the description, references to terms such as servers, client devices, applications and so on are for illustrative purposes only. The terms server and client device are to be understood broadly as representing computing devices with one or more processors and memory configured to execute machine readable instructions. The terms application and computer program are to be understood broadly as representing machine readable instructions executable by the computing devices.

FIG. 1 shows a simplified example of a distributed computing system 100. The distributed computing system 100 includes a distributed communications system 110, one or more client devices 120-1, 120-2, . . . , and 120-M (collectively, client devices 120), and one or more servers 130-1, 130-2, . . . , and 130-N (collectively, servers 130). M and N are integers greater than or equal to one. The distributed communications system 110 may include a local area network (LAN), a wide area network (WAN) such as the Internet, or other type of network. The client devices 120 and the servers 130 may be located at different geographical locations and communicate with each other via the distributed communications system 110. The client devices 120 and the servers 130 connect to the distributed communications system 110 using wireless and/or wired connections. The client devices 120 may include smartphones, personal digital assistants (PDAs), tablets, laptop computers, personal computers (PCs), etc. The servers 130 may provide multiple services to the client devices 120. For example, the servers 130 may execute software applications developed by one or more vendors. The servers 130 may host multiple databases that are relied on by the software applications in providing services to users of the client devices 120. For example, one or more of the servers 130 execute an application that implements the online service including the dialogue management system of the present disclosure.

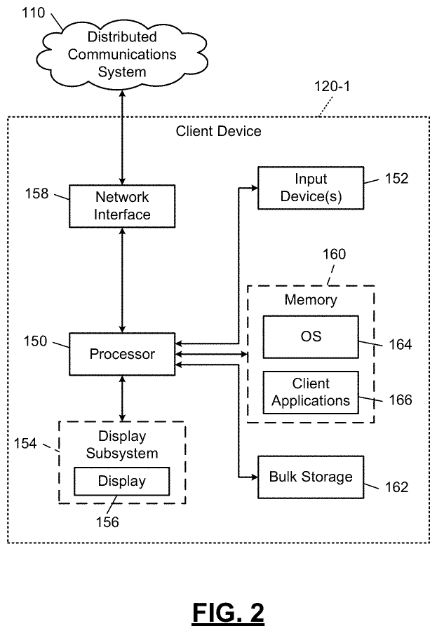

FIG. 2 shows a simplified example of the client device 120-1. The client device 120-1 may typically include a central processing unit (CPU) or processor 150, one or more input devices 152 (e.g., a keypad, touchpad, mouse, touchscreen, etc.), a display subsystem 154 including a display 156, a network interface 158, memory 160, and bulk storage 162. The network interface 158 connects the client device 120-1 to the distributed computing system 100 via the distributed communications system 110. For example, the network interface 158 may include a wired interface (for example, an Ethernet interface) and/or a wireless interface (for example, a Wi-Fi, Bluetooth, near field communication (NFC), or other wireless interface). The memory 160 may include volatile or nonvolatile memory, cache, or other type of memory. The bulk storage 162 may include flash memory, a magnetic hard disk drive (HDD), and other bulk storage devices. The processor 150 of the client device 120-1 executes an operating system (OS) 164 and one or more client applications 166. The client applications 166 include an application that accesses the servers 130 via the distributed communications system 110. The client applications 166 include an application that accesses the online service including the dialogue management system executed by one or more of the servers 130.

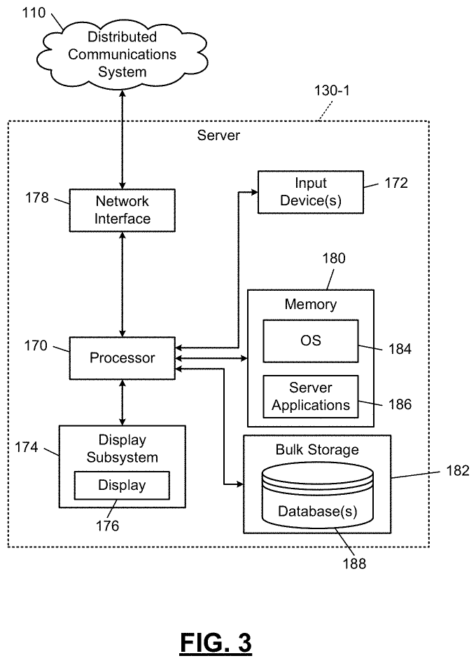

FIG. 3 shows a simplified example of the server 130-1. The server 130-1 typically includes one or more CPUs or processors 170, a network interface 178, memory 180, and bulk storage 182. In some implementations, the server 130-1 may be a general-purpose server and include one or more input devices 172 (e.g., a keypad, touchpad, mouse, and so on) and a display subsystem 174 including a display 176. The network interface 178 connects the server 130-1 to the distributed communications system 110. For example, the network interface 178 may include a wired interface (e.g., an Ethernet interface) and/or a wireless interface (e.g., a Wi-Fi, Bluetooth, near field communication (NFC), or other wireless interface). The memory 180 may include volatile or nonvolatile memory, cache, or other type of memory. The bulk storage 182 may include flash memory, one or more magnetic hard disk drives (HDDs), or other bulk storage devices. The processor 170 of the server 130-1 executes an operating system (OS) 184 and one or more server applications 186, which may be housed in a virtual machine hypervisor or containerized architecture, which include the online service and the dialogue management system of the present disclosure. The bulk storage 182 may store one or more databases 188 that store data structures used by the server applications 186 to perform respective functions.

The online service is a science-based online service and social community for engaging, learning and training the skills of happiness. The online service can be offered through a variety of computing devices including smartphones, tablets, laptops, etc. The online service is based on a framework developed by psychologists and researchers in the science of happiness, which includes positive psychology and neuroscience. The online service assists users in the development of many happiness skills such as, for example, Savor, Thank, Aspire, Give, and Empathize (or S.T.A.G.E.™). The online service includes an additional happiness skill called Revive that is concerned with physical wellness. Throughout the present disclosure, references are made to the STAGE skills for convenience only, and such references should be understood to include the sixth Revise skill. Each skill may be developed using various activities, ordered in increasing skill level, that gradually unlock as the user progresses in building that skill. Users of the online service may be given a range of activities from the STAGE skills, from reflective blogging and science-based games and quizzes, to real-life tasks that the users are asked to perform and report back on. Each activity is backed by scientific studies that are directly accessible by the user via links provided by the online service in the recommended activities.

The activities may be offered to users in several ways. Two examples described below are “Tracks” and “Personal Recommendation and a la Carte.” Tracks include sets of activities programmed to address a specific life situation or goal (e.g., “Cope better with stress; “Enjoy parenting more”, etc.) in, for example, a 4-week time period. Upon signing up with the online service, users may complete self-assessments that give them their initial happiness level as well as an initial recommended track. Users may complete approximately one part of a track each week, spanning 4 weeks altogether, for example. When users finish a track part, users may win, for example, a badge that represents their level of activity in that track part. Alternatively, the activities may also be offered as a personal recommendation and a la Carte. When not in a track, a user may be offered a personalized daily activity (an unlocked activity from a skill that the user has not accessed in the past week). Users also may pick activities from a skill menu and choose any unlocked activity.

As users perform their activities, users may create activity posts that are saved in their personal profile and build up a ‘digital happiness wallet’ they can reflect on. Posts may include the type of activity performed by the user, any text and images the user added, other people involved, if any, as well as the time and location for the post. When the activity is a conversation performed with the dialogue management system, a post may include a summary of record of the conversation. Posts also may appear on various activity feeds on the service, which allows other users to read, draw inspiration from, and offer encouragement in the form of comments and likes. Users may also follow activities posted by other users they find interesting if those users allow themselves to be followed or mark their post ‘public.’ Periodically, the online service may make suggestions for users to follow other users whose profiles match in terms of demographics and psychographics, as well as level of activity on the site and other criteria.

Users can keep track of their progress using periodic, scientifically-designed self-assessments that present them with their current happiness level compared to past levels. Over time, the online service may build a ‘Happiness Graph’ for each user, comprising of activities, people, places, and things correlated with the impact they had on the user's happiness levels. This information may be used to optimize the user experience and the activities the service suggests.

The following are some of the benefits and distinguishing features of the online service. For example, the benefits provided by the online service include, but are not limited to, the following: Clarity (e.g., 5 skills, level progression), Integrated Self Assessments (e.g., provides self-insights, Recommends tracks & activities), Progress Measurement (e.g., periodic happiness measurements allow the users to monitor their progress), Guided Experience (e.g., four week track experience optimizes habit formation, enables continued focus on a specific topic (e.g., parenting, stress)), Flexible (e.g., track structure allows the users to pick the activities and tasks they prefer from a wider selection of options), Personalized (e.g., activity recommendations are based on past user behavior and preference), Integrated Social Experience (e.g., users share and follow, like and comment on other users' posts), Increasingly Challenging (e.g., as the users progress, tracks require increased number of activities and higher level of challenge), Entertaining (e.g., variety of activity types, track content), Extendible in Several Dimensions (e.g., content: new tracks and track content (tasks, quizzes, polls etc.), activity types: adding new games and activity types, framework: adding new skills), and Multi screen (e.g., web, mobile accessibility).

The following are non-limiting examples of attributes that are unique to the online service compared to other digital well-being services. For example, the online service employs a Science-to-Action Framework (e.g., translation of the science of happiness into 5 skills, named activities per skill and actionable tasks per activity), provides Sustained Guidance (e.g., other feedback mechanisms either track external user activity with visually-limited feedback, or allow users to grow visual environments by interacting with them directly (and not use them to provide feedback on external activities)), provides Contextual Social Interaction (e.g., users socialize around contextual activity posts prescribed to others), provides Activity Variety (e.g., “One stop show” happiness service with real-life, reflective and gaming activities), provides Measure-Act-Measure loop (e.g., allowing users to track their progress as they go), and provides an efficient and versatile dialogue management system that uses a 3-tier architecture to facilitate dialogues about multiple activities performed by multiple users using the least amount of data structures.

The tracks, activities, and tasks offered by the online service are now described in further detail to enhance understanding of the dialogue management system. Tracks are sets of activities that are programmed together to address specific life situations, goals, or concerns that users have. A track name is actionable and concise (e.g., 5 words max). A track description (e.g., 140 words max) introduces the user to the track and explains what the user will achieve by completing the track. Each track is composed of four parts (described below; also see FIGS. 11A-11C). The number of activities and their level of difficulty increases as the user progresses from part 1 to parts 2, 3, and 4.

The following are examples of the rules that govern the tracks. Users have approximately one week to complete a track part and thus earn badges (regular or honors badge, depending on the number of activities they completed. Users are allowed to extend beyond a week and still win the regular badge. If a user reaches the regular badge threshold the user is allowed to ‘win’ the regular badge and move to the next part, or continue for the honors badge. This allows users to skip the remaining activities and win the regular badge if they prefer. Track activities can be ‘time-locked,’ queue-locked,′ or available. At start, for example, two activities are available for the user to perform, and one is ‘queue-locked,’ which means that if the user performs an available activity, it will make the ‘queue-locked’ activity become available. Each day, for example, three time-locked activities become ‘queue-locked,’ and queue-locked activities become available up to a limit of four available activities. This limit of four available activities is intended to avoid showing the users too many available activities when they next log in.

Every activity a user completes creates a post that is added to the user's profile. Users can mark their posts private (i.e., only visible to them and not visible to others) or viewable to other people (people who follow them and people doing the track in group mode with them). As part of social interaction, users can view the shared posts of other people who are following the track and can like or comment on them or follow the authors of those posts. Users can like and comment on posts to encourage each other and discuss their contents.

The online service offers some premium and expert tracks. These are special tracks created by experts and thought leaders in the field of emotional well-being and happiness science as premium tracks. The following provides a sample list of such tracks. The tracks fall under one of following life domains: Career & Money, Family & Kids, Leisure & Fun, Love & Intimacy, and Mind & Body.

‘Career And Money’ tracks include activities directed to the following aspects: Appreciate what I have (currently available), Reduce on-the-job stress, Get energized about my job, Stay upbeat while out of work, Balance work and home life, and Control my spending habits.

‘Family And Kids’ tracks include activities directed to the following aspects: Enjoy parenting more, Better cope with new parenthood, Better adjust to becoming an empty nester, Forgive and forget feud (with a family member), and Better cope with the stresses related to my aging parents.

‘Leisure And Friends’ tracks include activities directed to the following aspects: Be more socially connected, Talkers and listeners, Explore the Art in Happiness, Find more “me” time, and Be a better friend.

‘Love And Intimacy’ tracks include activities directed to the following aspects: Feel more loved by my partner, Feel and be more devoted to my spouse, Fight less and love more in my relationship, Find Mr. Right—or Mr. Right Now, Get over a broken heart, and Feel hopeful to start dating after divorce.

‘Mind And Body’ tracks include activities directed to the following aspects: Cope better with stress, Nurture my Body and Soul, Come to terms with getting older, Feel healthier, Be more optimistic about my potential, and Find more purpose and meaning in my life.

For example, each track includes four parts, each of which takes approximately one week for users to complete. If users run out of time, they have the option to extend their time by another week. Each part of a track includes a balanced mix of ‘reporter’ activities and ‘light’ activities. The reporter activities gradually increase in difficulty as a user progress through each of the four parts. Light activities include: Games (e.g., mini games, such as Hidden Object “mindfulness” game, training the user on a specific happiness skill), Quizzes (e.g., multiple-choice or true/false questions about a happiness topic), Activity Quizzes (e.g., users read a science paragraph about an activity and are quizzed with multiple-choice questions at the end), and Polls (e.g., polling users' opinion about a related topic and showing them community's vote breakdown). Reporter activities fall into two categories: “Essay” or “Do” activity, which asks users to reflect on a subject and make a log entry (e.g., reflective microblogging: users are asked to reflect on a topic and write down their thoughts (e.g., what they are grateful for, what they look forward to, taking another person's perspective, etc.)); and “Plan-Do” activity, which asks user to plan and perform an action in the real world, then come back and report on how it went (e.g., write about his/her experience (e.g., do a savoring exercise)). The conversational activities (i.e., the conversations performed with the dialogue management system) are different than reporter activities.

A mix of about 50% “reporter” activities and 50% “light” activities is used in each track part to avoid overwhelming the user. The online service allows for an activity to appear more than once in a track if it's a crucial activity for the track theme and there are new/different suggested tasks for each use. The number of activities per track part is flexible.

For example, a 7-day sequence of every track part includes a narrative purpose and a feel as if it has a beginning, middle, and an end that gives the user a sense of accomplishment. In the first days of a track part, the activities jump-start a key positive emotion the user will need for subsequent activities or asks the user to try something new, intriguing, fun, or funny—which rattles the user out of her funk and gets her in a good mood for what's next. In the middle of a track part, the activities build on (or complement) previous ones. An activity may be introduced that needs some extra thought or action. By day 4 or 5, the user feels a little more committed or motivated and willing to take on slightly more demanding activities. In the end, on the last day of a track part, users want something that's fun, easy or inspiring. Accordingly, unfamiliar/demanding tasks are avoided. The users anticipate a feeling of accomplishment but is intrigued enough to commit to the next part of their track.

The goal of the tracks is to create an appealing balance between activities that can be completed immediately by writing after a few minutes of reflection versus activities that require action (and in some cases, pre-planning) before reporting on how it went. In general, easier (levels 1 and 2) activities are programmed towards the beginning of a track (parts 1 and 2), and as a user progresses to the later parts of a track, the activities become more difficult (levels 4 and 5 activities), but this isn't required. Users are awarded badges based on how many activities they complete in each part of a track. The online service offers special badges for each part of a track.

Users interacting with the online service start off at level-1 in all skills. As they complete activities they progress in each skill from level-1 to level-2 etc. New activities, self-assessments, and other options unlock as the user reaches a higher level. For each skill, the online service offers relevant, science based activities that train the user in an entertaining way. As the users level up in a skill, they unlock new activities (Level 1 to level 5 activities are available in each skill). Each activity provides the user with several alternatives for completing the activity (“Suggested Tasks”) to pick from. Users can view an explanation of “why it works”: a short summary of the science behind that activity, complete with links to the actual study this activity is based on.

The STAGE framework of the online service captures the essence of the science of positive psychology and allows for presentation to mainstream consumers in an accessible way. The STAGE framework of the online service offers different types of science-based activities to users. The online service provides nearly sixty science-based activities in various tracks to help users build the following five essential happiness skills: (1) Savor—Noticing the goodness around you and taking time to prolong and intensify your enjoyment of the moment. Savoring can involve the past (reminiscing), the present (mindfulness), or the future (positive anticipation); (2) Thank—Practicing gratitude; identifying and appreciating the things we have and the people in our lives; (3) Aspire—Feeling hopeful, having a sense of purpose and meaning in our lives, being optimistic; (4) Give—Performing acts of kindness; being generous and forgiving; and (5) Empathize—Imagining and understanding the emotions, behaviors, or ideas of others; having compassion. See FIGS. 10A-10N for details.

The framework of the online service provides 2-3 suggested tasks for each activity. For example, once the “reporter” activities are determined for each track part, the online service provides 2-3 suggested tasks for each activity. These tasks retain the essence and the science of the proven intervention activity, but make sense within the theme of the track. The tasks are fun, and yet give clear and concise directions. A user needs to pick one of these tasks to complete in order to get credit for the activity. That is, users only need to complete only one of the task options in order to get credit for a given activity. When a user selects an activity, s/he can choose one of the two suggested tasks or a third “You Decide How” (YDH) option. Each suggested task is accompanied by a “Why It Works” section, which includes science references and explains why the activity is useful and how it relates to happiness. Below are some examples of sample activities and suggested tasks. A comprehensive list of tracks and activities is provided in a table shown in FIGS. 10A-10N. An example of a track and its activities and tasks is shown in a table in FIGS. 11A-11C.

For example, for the track Feel More Loved by My Partner, and activity Today's Grateful Moment [Skill: Thank], a Suggested Task #1 may include the following. Name: The Little Stuff Counts (e.g., think of the reason you first fell in love with your partner or spouse—a trait or characteristic he/she still holds today. It could be his sense of humor, her kind generosity, or maybe his sex appeal. Write down some thoughts and spend a minute appreciating those same traits today). A Suggested Task #2 may include the following. Name: Thanks, Partner! (e.g., think of one good thing that happened today involving your partner or spouse. Write it down and add a few details about how it made you feel and the role you played, if any, in the positive experience). A You Decide How (YDH) task may include the following. For example, think of something, great or small, that you feel grateful for and describe it in a few words. Add a photo too if desired.

FIG. 4 shows a block diagram of the online service described above, which is shown as the online service 200. The online service 200 comprises a content management system (CMS) 202, a plurality of modules 204 controlling various features and aspects of the online service 200 described above, and a plurality of databases 206 associated with and utilized by the respective the plurality of modules 204 and the CMS 202. The CMS 202 manages the overall content provided by the online service 200 to the users of the online service 200 using the plurality of modules 204 and the plurality of databases 206.

The plurality of modules 204 comprises an authentication module 210, a skill assessment module 212, a track prescribing module 214, a post sharing module 216, a follower managing module 218, a graph generating module 220, and a dialogue management module 230. The authentication module 210 establishes user accounts and controls the users' access to the online service 200. The skill assessment module 212 assesses a user's skills initially when the user signs up and later periodically as the user performs the prescribed activities. The track prescribing module 214 prescribes the tracks and modifies the tracks to the users according to their skill assessments as described above. The post sharing module 216 manages publication of the posts shared by the users (e.g., keeping them private or publishing them depending on the users' preferences, handling the likes and comments on the posts by other users, etc.). The follower managing module 218 manages the follower recommendations to the users based on profile matching as described above. The graph generating module 220 generates the happiness graphs as described above. The dialogue management module 230 conducts dialogues between the users and the online service 200 and includes the dialogue management system as described below in detail.

The plurality of databases 106 comprises a database for each of user profiles 240, tracks 242, activities 244, tasks 246, assessments 248, posts 250, graphs 252, content 254, and research data 256. The online service 200 provides content to the users of the online service 200 using the plurality of modules 204 and the plurality of databases 206 under the control of the CMS 202.

FIGS. 5A and 5B show the dialogue management system 230 in further detail. FIG. 5A shows the dialogue management system 230 having a 3-tier or 3-layer architecture. FIG. 5B shows an example of a dialogue box (or a dialog box) 270 on a user's computing device (e.g., a client device 120-1 shown in FIGS. 1 and 2). Throughout the present disclosure, the various “dialogue files” can also be called the respective “dialog files.”

In FIG. 5A, the dialogue management system 230 includes a single master dialogue file (also called a master file or a master) 232, and a plurality of skeleton dialogue files 234-1, 234-2, . . . , and 232-N, where N is the number of activities 244 (e.g., N is nearly 60) (collectively called skeleton dialogue files, skeleton files, or skeletons 234). For each of the skeleton dialogue files 234, the dialogue management system 230 includes a plurality of skin dialogue files 260-1, 260-2, . . . , and 260-M (collectively called skin dialogue files, skin files, or skins 260). The skin dialogue files 260 include You Decide How (YDH) skin files and task skin files. Throughout the present disclosure, an individual skin file (YDH or task), a YDH skin file, and a task skin file are also referenced by the numeral 260. The dialogue management system 230 and its components, which include the master dialogue file 232, the skeleton dialogue files 234, and the skin dialogue files 260 are now described below in further detail.

The dialogue management system 230 allows the users to engage in having a brief dialogue with the online service 200 about an experience emanating from performing a prescribed activity 244. Dialogue boxes are generated using a tiered system of files, each with a unique purpose (see an example of a dialogue box shown in FIG. 5B). Specifically, the dialogue boxes are created using three sets of tiered or layered files: a single master dialogue file (master) 232, a plurality of skeleton dialogue files (skeletons) 234, and a plurality of skin dialogue files (skins) 260. Accordingly, the dialogue management system 230 that creates the dialogue boxes includes three layers of files—master, skeleton, and skin (MSS)—and can also be called a MSS system. Note that theoretically there can be multiple master files 232; however, practically, having a single master file 232 simplifies the design of the dialogue management system 230.

While a track 242 includes many activities 244 and each activity 244 includes many tasks 246, the dialogue management system 230 includes a hierarchical architecture that leverages some amount of overlap that exists across the activities 244. The dialogue management system 230 includes a single master file 232 for all the activities 244, one skeleton file per activity 244, and one skin file 260 per task 246. The master dialogue file 232 includes the entire and complete markup language or Script based structure that is needed to run any dialogue (i.e., for any activity 244 and any task 246). For example only, the master dialogue file 232 can be a JavaScript Object Notation (JSON) file or an Extensible Markup Language file. The dialogue management system 230 includes only one master dialogue file 232. The master dialogue file 232 represents the full set of capabilities of the dialogue management system 230. The texts in the prompts, buttons, choices, and responses in the master dialogue file 232 are generic. For example, in the master dialogue file 232, a response after a user makes a single choice might be “Response to first choice.” This allows the master dialogue file 232 and its CHTML based structure to work in any context for any activity 244.

A skeleton dialogue file 234 represents the specific structure for an activity 244 (e.g., a skeleton can be designed for S-01 Savor the Small Stuff). The skeleton dialogue file 234 is a JSON file that makes selected references to the CHTML structure in the master dialogue file 232 through the use of “include” statements.

A skin file 260 (i.e., one of the skin files 260 corresponding to the skeleton file 234 associated with the activity 244) represents the actual text to be presented when running a skeleton dialogue file 234 as well as the specific names for variables called life graph variables (LGVs) to be saved for a skeleton dialogue file 234. A skin file 260 is a spreadsheet or a comma separated value (CSV) file that specifies the location of each string of text and the specific text to be used in a dialogue.

The dialogue management system 230 includes two layers of skins 260. Every skeleton dialogue file 234 has an associated overview or You Decide How (YDH) skin file 260. Additionally, a task skin file 260 can also be assigned to a specific task 246 (e.g., there would be a specific task skin 260 for S-01-T-27 Smell the Roses).

Running a dialogue requires identifying a skeleton dialogue file 234 (for example, the skeleton for S-01 Savor the Small Stuff) and a skin file 260 (for example, the skin for S-01-T-27 Smell the Roses).

A dialogue can be initiated in two ways. In a first way, the master 232, the skeleton 234, and the skin 260 can be combined or compiled either offline in the CMS 202 or in runtime on demand at the time of invocation of the dialogue. The advantage of the former way is that the availability of a full development environment allows the CMS 202 to manage different versions of each master 232, the skeleton 234, and the skin 260 and identify and debug errors if compilation fails.

More specifically, the master dialogue file 232 is typically a single file. For example, only one version of the master dialogue file 232 may exist on the server (i.e., in the online service 200) at a given time. The master dialogue file 232 can be edited and updated over time (e.g., via the CMS 202), but in ways that overwrite the prior version. The master dialogue file 232 includes all of the core logic needed to determine and lay out the flow of any dialogue that can occur on the dialogue management system 230. The master dialogue file 232, therefore, is comprehensive and non-specific.

For example, the master dialogue file 232 includes the code necessary to run any language modeling and analysis algorithms, performing tasks such as the natural language classifiers (NLCs), Named Entity Recognition, Sentiment Analysis, and Linguistic Style Analysis and Transformation. For example, such algorithms include but are not limited to machine learning, deep learning, neural networks, statistical pattern recognition, semantic analysis, linguistic analysis, and generative models. A final user-facing dialogue may rely on the analysis of user input (e.g., one or two NLCs).

Every potential choice point that can occur in the flow of a dialogue is coded into the master dialogue file 232. The master dialogue file 232 includes placeholder text that is very broad and generic (e.g., “Response to user”; or e.g., choices for the user can be “Choice 1” and “Choice 2”). Alternatively, the default text, where breadth is not required, can be specific, such as ending the dialogue with “Goodbye” or offering the user choices such as “Yes” and “No”.

Skeletons 234 and skins 260 (i.e., the skeleton dialogue files 234 and the skin dialogue files 260) are where specific conversations and interactions with the user are designed. The dialogue management system 230 includes a skeleton dialogue file 234 for each core activity 244 offered to the users (e.g., the online service 200 includes nearly 60 activities). A skeleton dialogue file 234 is decisive, singular manifestation of the conversation flow offered by the master dialogue file 232. For example, if the objective is to interview the user about a relationship with a person in the user's life and the user's favorite things about that person, the skeleton dialogue file 234 for this interview can clearly delineate the flow for this conversation. The flow in the skeleton dialogue file 234 is deterministic, such that a series of given inputs from the user create a specific, exact conversation with the dialogue management system 230. However, the flow in the skeleton dialogue file 234 is dynamic, and a different set of user inputs can create a different conversation with the dialogue management system 230.

A skeleton dialogue file 234 may utilize only a small portion (e.g., 20% or 10%) of the dialogue portions or sub-dialogues defined in the master dialogue file 232. A skeleton dialogue file 234 may also use the dialogue portions of the master dialogue file 232 more than once. No specific text is determined by the skeleton dialogue file 234. So the skeleton dialogue file 234 can carry over the default text defined by the master dialogue file 232.

Furthermore, there can be an overlap between some of the activities 244. In such instances, the skeleton dialogue files 234 for such overlapping activities 244 can utilize the same or similar dialogue portions of the master dialogue file 232. Further, these dialogue portions in the master dialogue file 232 themselves can be reduced in number based on the overlap in some of the activities 244, which results in optimization in the design of the master dialogue file 232 and which provides additional synergy between the skeleton dialogue files 234 and the master dialogue file 232.

A skin dialogue file 260 (i.e., each one of the skin dialogue files 260) includes a list of “specifics” which describes the exact sentences and phrases to be used by the dialogue management system 230 at each point in the conversation flow described by a given skeleton dialogue file 234. Skin dialogue files 260, therefore, are inherently tied to a specific skeleton 234 and are not paired with other skeletons 234. The dialogue management system 230 includes a skin dialogue file 260 for each specific task 246 for an activity 244 offered to users by the online service 200. For example, for the nearly 60 core activities, the dialogue management system 230 includes anywhere from dozens to hundreds of skin dialogue files 260 for each activity 244.

In some cases, the default text in the master dialogue file 232 can suffice, such as giving the user a choice between “Yes” and “No”. In these cases, the skin dialogue file 260 can include an indication such as a null entry, allowing the text to be determined by the master dialogue file 232. If the master dialogue file 232 is subsequently changed so that these choices respectively become “Absolutely” and “No way,” these changes are automatically reflected in any conversation where the skin dialogue file 260 has null entries at these points. For the most part, however, the skin dialogue files 260 determine the response text, and the skin dialogue files 260 often overwrite the default responses of the master dialogue file 232.

Every skeleton dialogue file 234 has paired with it a You Decide How (YDH) skin dialogue file 260 that is designed in a broad, general way depending on the scope of the conversation determined by the skeleton dialogue file 234. For example, if a savoring skeleton dialogue file 234 is built to help the user savor a positive feeling, the YDH skin dialogue file 260 can determine all the sentences and phrases for this conversation. However, a new skin dialogue file 260 may be created from this YDH skin 260 that focuses the user specifically on savoring food. A different skin dialogue file 260 may be created from this YDH skin 260 that focuses the user specifically on savoring an experience. Notably, due to the tiered architecture of the dialogue management system 230, no changes are required at the master 232 or skeleton 234 level to add this new activity. The only edits needed are to the YDH skin dialogue file 260, where any new phrases or guidance specific to food (or experience) can be added or edited. This new skin dialogue file 260 can then be paired with the savoring skeleton 234 to run a food (or experience) savoring conversation. Due to the tiered architecture of the dialogue management system 230, this versatility is accomplished without requiring code changes at the master 232 or skeleton 234 level. This significantly simplifies the design of the dialogue management system 230.

The master dialogue file 232 can offer a broadly-defined capability to identify an object of the conversation. The master dialogue file 232 includes the built-in architecture (CHTML based data structures) to receive variables that can decide how the object is identified, how many questions are asked of the user, whether or not to provide a response at certain points, etc. The skeleton dialogue file 234 is where the flow-determining variables that are fed to the master dialogue file 232 are defined. Accordingly, the result of designing a skeleton dialogue file 234 is the decision to use the identify capability to ask two questions, for example, and respond any time the user identifies an emotion or an activity 244 based experience. The skin dialogue file 260 paired with the skeleton dialogue file 234 defines, among all of the dialogue's specific text, the questions that can be asked, which for one particular skin dialogue file 260 may be “What is your favorite hobby?” and “How do you feel when you are engaging with this hobby?”. The skin dialogue file 260 paired with the skeleton dialogue file 234 additionally defines the full set of potential responses to emotions that might be provided in the answer by the user.

The master dialogue file 232 includes a library of sections or dialogue portions, each of which is a subset (or sub-dialogue) of a conversation that is focused on a single task 246 and includes distinct pieces of a conversation designed to achieve a goal in the conversation. Only a few of the dialogue portions are used during a dialogue. Further, some of the same dialogue portions may be used in combination with other dialogue portions in another dialogue. Essentially, for conducting a dialogue about an activity 244, a few of the dialogue portions from the master dialogue file 232, a skeleton dialogue file 234 corresponding to the activity 244, and a plurality of skin dialogue files 260 corresponding to the tasks 246 associated with the activity 244 are compiled together.

The dialogue management system 230 conducts the dialogue with the user in a versatile, life-like manner using the compiled combination of the dialogue portions from the master dialogue file 232, the skeleton dialogue file 234, and the skin dialogue files 260. This method of conducting dialogues eliminates the need to have a one to one correspondence between the number of dialogue portions of the master dialogue file 232 and the number of activities 244. For example, the dialogue management system 230 may include as few as 18-20 sections for as many as 60 activities and a much greater number of tasks 260. Accordingly, this method, comprising generic, modular, and reusable data structures designed in the master file 232, which are then selected by the skeleton 234 and modified by the skins 260, results in significant improvements and optimizations in the architecture and resource utilization of the databases of the online service 200.

In a conversation (i.e., in a dialogue), a node is an atomic element. A node typically includes a prompt for the user and includes logic to process the user's response to the prompt. The prompt and the user's response (user input) can include one or more of text, speech/audio, and video including virtual reality (which can be used to extract body posture/positions facial expressions etc. for use as user input). Based on the processing of the response, the conversation moves to a next node. A section or dialogue portion in the master file 232 includes a group of nodes.

There are two types of sections in the master file 232: linear (or sequential) sections and adherence sections. The nodes in the sequential sections are processed sequentially (i.e., a next node is processed when a condition is satisfied after processing a prior node). In an adherence section, after a node is processed, control always returns to the first node, and a check is performed as to which, if any, variable remains to be filled, and control moves to that node for which a variable needs a response. The process is repeated until all the variable are filled or until a counter expires. In case of a non-ending loop (e.g., due to repeated irrelevant responses from the user), a counter is maintained, and the loop is exited on expiration of the counter. The counter is only an example; instead, any other stopping condition that is guaranteed to be met within a reasonable number of conversation turns can be used.

Across the different sections or dialogue portions of the master 232, while the prompts may be different, and the content of the text (in the user response) may be different, the structure of the sections is not very different across different activities 244. For example, in a conversation, regardless of the activity 244, the dialogue may start with a greeting and may end with a summary, both of which can be short, repeatable (i.e., reusable) sequential sections. The dialogue may additionally include an adherence section to elicit responses for a few variables needed to conduct the dialogue. The dialogue may further include another section to clarify or disambiguate an item, for example.

These sections tend to have similar structures though different content. Further, irrespective of the number of activities 244 offered by the online service 200, these sections of the master file 232 are few in number (i.e., they are not as many in number as the number of activities 244; or there is no one to one correspondence between the sections of the master file 232 and the activities 244). Accordingly, the master file 232 includes only a handful of sections and is a collection or an array of a few sections that (can but) do not include any specific content (e.g., what to ask), but have variables with generic values that can be and are usually overwritten by the skeleton 234 and the skins 260.

The skeleton file 234 simply contains a series of include calls that select a few sections (dialogue portions) from the master file 232 to accomplish the dialogue at hand. At this point, however, the dialogue management system 230 does not know the exact nature of the dialogue (e.g., whether the user wants to savor an experience or food). The skeleton 234 therefore also includes an identify section from the master file 232, which is very generic in nature (e.g., it can identify a person, an object, etc.).

The values for the variables in these sections are provided by the skin file 260. These values are elicited from the user by the skin 260 by prompting the user with questions (e.g., multiple choice questions). The YDH skin file 260 is also general in nature (e.g., it can indicate savoring something but cannot further specify an experience or food). The task skin 260 provides the specific values for the variables that override the generic values of variables as well as specific values provided by the master file 232, if any. These features of the master file 232, the skeleton files 234, and the skin files 260 eliminate the need for providing custom dialogue scripts by anticipating every input from users, which again greatly simplifies the design of the dialogue management system 230.

The specific features or data structures employed by the master 232, the skeletons 234, and the skins 260 are now described. Throughout the remainder of the disclosure, while references are made to natural language classifiers (NLCs) and associated variables and values, NLC is used only as an illustrative and non-limiting example of a task performed by language modeling and analysis algorithms mentioned above.

The master dialogue file 232 includes the following features or data structures that are implemented in markup language or Script: conditional values, default NLC values, and a single array. In the conditional values features or data structures, as part of a variable/value pair, a capability to assign values based on a condition is provided (e.g., response_text can be assigned to a string based on the value of_emotion). For the first condition that evaluates as true, the variable assignment is made, and no further conditions are evaluated. Unless defined, by default the “else” condition is equal to the current value of the variable (e.g., in the above example, the “else” value can be “_response_text”).

In the default NLC values features or data structures, as part of the initial attributes of a section within the Script, included is an attribute named “nlc_defaults” which specifies what the output of a classifier should be depending on whether a classifier is used or not. Each classifier used in a section (dialogue portion) is identified by name and a default value is defined. If a classifier is present in a section (dialogue portion) and a default is not defined under nlc_default, the default value is a blank string.

In the single array of variables feature or data structure, for each choice within a single (or multi) input request, three attributes are defined: a “label”, an “Igv_value”, and a “prompt”, with each choice identified by a “name” to the left of the colon, and the three attributes as strings defined to the right of the colon. The first attribute, “label”, is the text that should be presented as a choice to the user. The following two attributes are accessible as attributes of sensor objects after a selection is made. Accordingly, an Igv_value(sensor) is an Igv_value text of a choice that is made, and a prompt(sensor) is a prompt text of the choice that is made. In other words, to illustrate, if a user choses a third option, for example, Igv_value(sensor)==‘third choice text’ and prompt(sensor)==‘Response to third choice’. If the “label” of a choice is blank, then that choice is not presented. If every choice has a blank label, a validation error should occur (however, this happens at the level of the skeleton 234 and skin 260; the master 232 allows for all blank values that should be filled in at the skeleton/skin level).

The skeleton dialogue file 234 contains “include” calls for selected dialogue portions from the master dialogue file 232, including both variable folders, global handlers, and sections (dialogue portions). The following feature or data structure are implemented for the skeletons: NLC Switches, Variable Assignments, and Section-to-Section Flow. In the NLC switches features or data structures, as an attribute of an included section (dialogue portion) in the master 232, “nlc_active” defines whether a classifier is run or not in that section (dialogue portion). The “nlc_active” attribute defined in the skeleton works in conjunction with the “nlc_default” attribute defined in the master dialogue file 232. When “nlc_active” for a classifier is set to false, the output of the classifier is the default defined in “nlc_default”. By default, each classifier present in an included section (dialogue portion) has an “nlc_active” value of false. So unless the skeleton dialogue file 234 defines an NLC as active (set to true), that classifier will not run in this section (dialogue portion).

In the variable assignments features or data structures, as an attribute of an included section (dialogue portion), “assign” redefines values for certain variables found in that section (dialogue portion). For any variable present in the section (dialogue portion) and not included in the “assign” list, the value remains as it is defined by the master dialogue file 232. However, the “assign” values made by the skeleton dialogue file 234 override the values set by the master dialogue file 232. Functionally, the assign values help define the flow and structure of an included section (dialogue portion), allowing importing a single block of code that can be used differently depending on the value of these variables. This feature is not merely better code but rather a better data structure architecture that yields efficiencies in database design and resource usage and significantly improves the functioning of the databases as one skilled in the art can appreciate.

The section-to-section flow feature or data structure is as follows. The master dialogue file 232 has “next”/“goto” statements that reference every section (i.e., dialogue portion) within the master dialogue file 232. When a skeleton dialogue file 234 includes only a subset of the sections (dialogue portions) from the master dialogue file 232, references to those sections (dialogue portions) that are not included in the skeleton dialogue file 234 need to be handled. The master dialogue file 232 includes three “identify” sections (dialogue portions) named “identify”, “2nd_identify”, and “3rd_identify”. For example, a given skeleton dialogue file 234 may include only the “identify” and “2nd_identify” sections (dialogue portions). In the “2nd_identify” section (dialogue portion), the master dialogue file 232 has “next”/“goto” statements pointing to “3rd_identify”, which does not exist in this skeleton dialogue file 234 in this example. At runtime, this skeleton dialogue file 234 should simply move to the identified section (dialogue portion) in the master dialogue file 232 (the “3rd_identify” section or dialogue portion in this example) and then look sequentially section by section for the next section or dialogue portion that the skeleton dialogue file 234 actually does include.

In the skin dialogue files 260, there are two levels of skins. A YDH (or overview) skin, and a task skin. The skin dialogue file 260 can be in a spreadsheet format but can ultimately run as a comma separated value (CSV) file in the content management system (CMS) 202 of the online service 200. First few top rows under the headers rename the life graph variables (LGVs) used by the skeleton dialogue file 234. For every instance of the LGV name in the “Original” column, it is replaced with the name in the “Value” column across the entire skeleton dialogue file 234. If an LGV in the skeleton dialogue file 234 is either not referenced here or has a blank value in the “Value” column, the original name persists. Subsequent rows redefine the text of the skeleton dialogue file 234. The text in the “Original” column is a reference to the text in the master dialogue file 232 at that location. The “Value” column is the new text that replaces the existing text from the master dialogue file 232. If the “Value” column is blank, the value from the master dialogue file 232 persists. But the priority is given to the skin 260. Ideally, the YDH skin 260 can be automatically generated from a skeleton dialogue file 234 in the CMS 202 by identifying every LGV and every segment of text. An exported skin created by the CMS 202 would have an empty “Value” column. An “Author” column designates whether or not this row is to be included in an automatically generated task skin 260. A “0” indicates it is not included, and a “1” indicates that it is included.

The task skin 260 can be automatically generated from the YDH skin 260 by: (1) removing the rows with “Author” designated as “0” and then removing the “Author” column altogether; (2) assigning each “Value” entry of the task skin 260 as the “Value” entry of the YDH skin 260 if it's not empty or the “Original” entry of the YDH skin 260 if the “Value” entry is empty; (3) creating an empty “Value” column; and (4) adding a “Legacy” column with one cell automatically populated with the “Short text”, “Description text”, and “Short text labels” already in the CMS 202 for the designated task 246. For each of these legacy task attributes, a tag is present that defines and separates the different strings. The “Value” column can then be filled in. When the CMS 202 is running an activity 244 using a task skin 260, it first prioritizes the “Value” entries from the task skin 260; if those are empty, next prioritizes the “Value” entries from the YDH skin 260; and if those are also empty, lastly prioritizes the “Original” entries from the YDH skin 260. If all of these values are blank for an “ask”/“prompt” or “next”/“text” entry, the dialogue management system 230 does not create a text bubble and continues with the flow of the dialogue. As described above, if the value for a single/multi label is blank, then it is not shown, and if all the labels for a single/multi input are blank, there is a validation error. The task skin file 260 is still paired with the original skeleton dialogue file 234. Accordingly, for example, to run S-01 Savor the Small Stuff in “You Decide How” mode, the dialogue management system 230 pairs the S-01 skeleton dialogue file 234 with the S-01 YDH skin file 260; to run S-01-T-27 Smell the Roses, the dialogue management system 230 pairs the S-01 skeleton dialogue file 234 with the S-01-T-27 task skin file 260; and so on.

In FIG. 5B, the user initiates the dialogue 270 (e.g., using a drop down menu from the online service 200), which is presented on the user's device (e.g., client device 120-1) in the form a user interface (UI). For example, the dialogue box 270 can appear similar to the UI of a text messaging app on a smartphone. In the dialogue 270, the entity “Service” represents an automated conversational agent driven by the 3-tier architecture of the dialogue management system 230 described above.

The dialogue 270 can begin with a greeting. The dialogue 270 can end with a summary and/or another greeting. The dialogue 270 provides the online service 200 (via the dialogue management system 230) another opportunity, in addition to the tracks 242, activities 244, and tasks 246, to effect an intervention, for example, by coaching the user on a particular happiness skill such as how to practice empathy or how to improve practicing empathy. The dialogue 270 also offers the user the opportunity to share his or her experience, exhibit his or her skill level regarding a particular happiness skill via the dialogue 270, and improve the particular happiness skill based on the coaching received from the online service 200 via the dialogue 270.

While not shown, the dialogue 270 can include text message as well as audio/video messages from either or both of the service and the user. Further, the dialogue can also include graphics such as emoticons, photos, videos, music, and so on that can be exchanged by and between the service and the user (i.e., either or both of the service and the user can also provide the graphics such as emoticons, photos, videos, music, and so on).

FIG. 6 shows a method 300 for conducting a dialogue between the online service 200 and a user of the online service 200 using the dialogue management system 230. For example, the method 300 is performed on one of the servers 130 and includes presenting the dialogue 270 on a user device such as the client device 120-1 via the distributed communications system 110.

At 302, the method 300 checks whether a user is initiating a dialogue 270 with the online service 200. At 304, if a user initiates a dialogue 270 with the online service 200, the method 300 receives an initial input from the user. At 306, based on the user input, the method 300 determines an activity 244 that the user wants to discuss in the dialogue 270 and identifies a skeleton file 234 for the activity 244. At 308, the method 300 identifies a skin file 260 for a task 246 associated with the activity 244. At 310, the method 300 includes dialogue portions from the master file 232 selected based on the activity 244 to conduct the dialogue 270. At 312, the method 300 combines the selected dialogue portions of the master file 232, the skeleton file 234 for the activity 244, and the skin file(s) 260 for the task 246 (e.g., the method 300 compiles these master 232, skeleton 234, and skin 260 elements). At 314, the method 300 generates a dialogue handler generated based on the combination or compilation that is used to conduct the remainder of the dialogue 270.

At 316, the method 300 receives additional inputs from the user. At 318, the method 300 conducts the dialogue 270 with the user based on the user inputs using the dialogue handler (e.g., the method 300 interactively responds to the user inputs). At 320, the method 320 determines if the user wants to end the dialogue 270. The method returns to 316 if the user wants to continue the dialogue 270. Otherwise, the method 300 ends.

FIG. 7 shows a method 400 for designing and generating the master file 232. At 402, the method 400 creates a library of dialogue portions such that the number of dialogue portions is less than the number of activities 244 (i.e., there is no one to one correspondence between the number of dialogue portions of the master file 232 and the number of activities 244 offered by the online service 200). For example, the method 400 identifies and takes advantage of any overlap or redundancies across the activities 244 offered by the online service 200.

At 404, in the library of dialogue portions, the method 400 creates a standard greeting dialogue portion to be presented at the beginning of in any dialogue 270 irrespective of underlying activity 244, and a standard summary dialogue portion (or another standard greeting dialogue portion) to be presented at the conclusion of any dialogue 270 irrespective of underlying activity 244. At 406, the method 400 designs variables with generic values (and a few variables with specific values) in the dialogue portions of the master file 232. At 408, the method 400 designs or configures the generic variables to accept specific value assignment from skeletons 234 and skins 260. At 410, the method 400 designs a plurality of the dialogue portions of the master file 232 to include sequential nodes. At 412, the method 400 designs or configures a plurality of the dialogue portions of the master file 232 to function or operate as adherence dialogue portions.

FIG. 8 shows a method 440 for designing and generating skeleton files 234. At 442, the method 440 creates a skeleton file 234 for an activity 244 (i.e., the method 440 creates one skeleton file 234 per activity 244 offered by the online service 200). At 444, the method 440 provides “include” calls in the skeleton file 234 to select relevant dialogue portions from the master file 232. At 446, the method 440 provides variable assignments to the selected dialogue portions based on user input to conduct the dialogue between the user and the online service 200. At 448, the method 440 provides section to section flow handling to conduct the dialogue between the user and the online service 200. For example, the order in which the flow of or between the sections is conducted during a dialogue may be different than the order in which the sections are arranged in the master file 232.

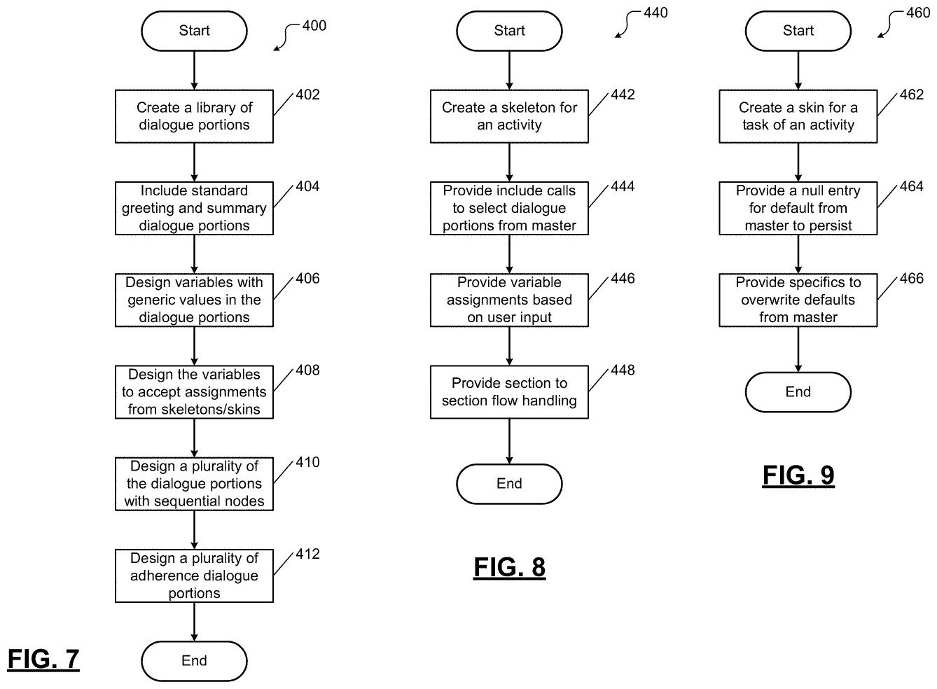

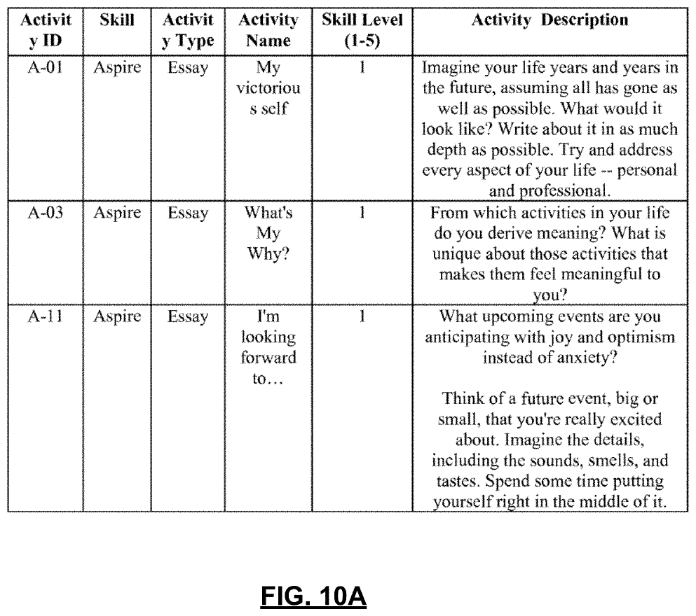

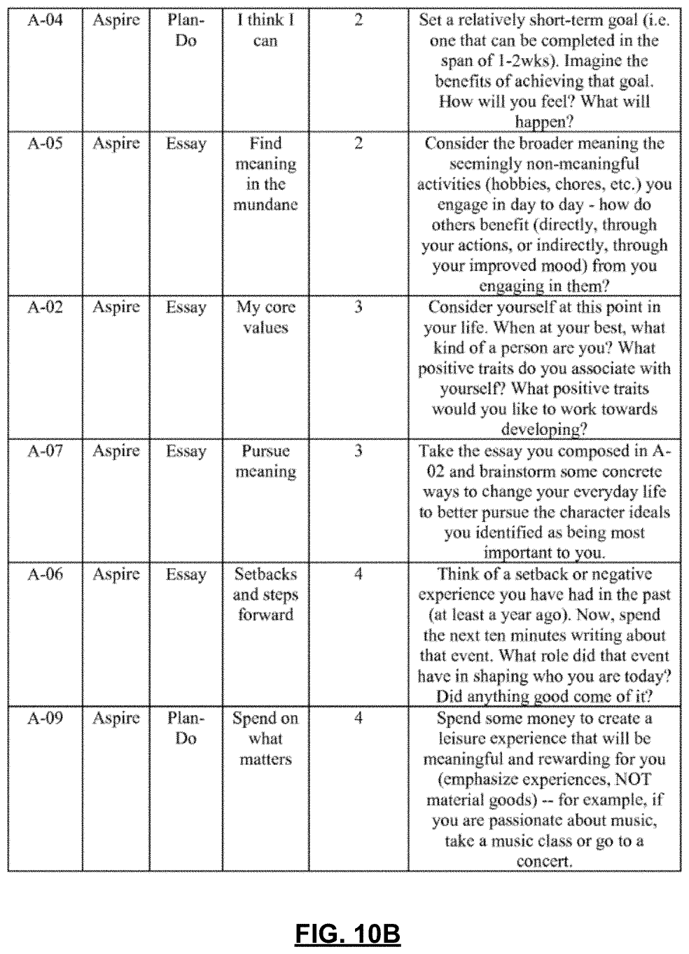

FIG. 9 shows a method 460 for creating a skin file 260. At 462, the method 460 creates a skin file 260 for a task 246 for an activity 244 (i.e., the method 460 creates a skin file 260 for each task 246 of an activity 244 offered by the online service 200). At 464, the method 460 provides an indicator such as a null entry to allow for a default value for a variable from the master file 232 to persist. At 466, the method 460 provides a specific value to overwrite a default value for a variable from the master file 232. The specific value is based on the user input and is passed to the skeleton file 234, which then assigns it to a suitable variable in a selected dialogue portion from the master file 232.

The dialogue management system 230 of the present disclosure differs from a chatbot. A chatbot is a very general description of any conversational agent that communicates with a user via text or voice/video on a turn by turn basis. A chatbot can therefore be intelligent (e.g., use machine learning) or completely pre-scripted; so it is very broad in scope. The differences between the dialogue management system 230 of the present disclosure and a chatbot are in the specific applications and its 3-tier architecture based on the specific applications. The dialogue management system 230 does not focus on delivering efficacious psychological interventions in the best possible way, and on using machine learning and dialogue management mechanisms to accomplish that. Rather, the dialogue management system 230 is an efficient way to create and program a “chatbot” using the 3-tier architecture described above so that the scripts governing the dialogues do not have to be created for all possible conversational scenarios and so that the scripts governing the dialogues can reuse some code.

Further, the dialogue management system 230 of the present disclosure differs from other automated customer support systems. Specifically, the difference is due to the operation of the dialogue management system 230 based on the tracks 242, the activities 244, and the tasks 246, where the activities 244, about which dialogues are conducted, are recommended by the online service 200. This schema of the online service 200 creates a unique opportunity for designing the synergistic 3-tier architecture to conduct dialogues as described above. Unlike the online service 200, systems that do not evaluate feedback from users regarding activities recommended by the systems and that do not attempt to improve user behavior via interventions offered based on the feedback, naturally lack the need for the 3-tier architecture described above. Of course, the dialogue management system 230 can be used with any other system that evaluates feedback from users regarding activities recommended by the system and that attempts to improve user behavior via interventions offered based on the feedback.

In sum, the dialogue management system 230 of the present disclosure uses a novel 3 layer approach—a generic master file 232 that can cater to dialogues on any of the nearly 60 activities offered by the online service 200, a skeleton file 234 that is specific per activity 244 and that links to one or more “sections” or dialogue portions in the master file 232 (some of which can be reused for another activity 244), and a plurality of skin files 260 that handles the input and output at the user interface presented to the user as a dialogue box 270. For each dialogue 270, these 3 elements are combined and a dialogue 270 is conducted. For another user or another activity 244, another combination is used to conduct another dialogue 270. The synergy provided by the 3 tier approach is that the generic nature of the master file 232, the ability of the skeleton file 234 to include sections of the master file 232 in any combination as needed, and the ability of the skins 260 to provide the specific values to variables in the selected sections of the master file 232 result in significant reuse of the sections of the master file 232, which yields efficiencies in database design and use of database resources. The dialogue management system 230 is versatile in that it works across all activities 244 offered by the online service 200 and regardless of the variations in the user's inputs and in the activities 244. Thus, the 3 tier design of the dialogue management system 230 improves the functionality of the computer databases 206, not merely code.

The foregoing description is merely illustrative in nature and is not intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A system for conducting dialogues with users of an online service recommending N activities for enhancing mental health of the users, where N is an integer greater than 1, the system comprising:
a processor; and
memory storing instructions which when executed by the processor configure the processor to:
receive, at the system, an input from a user via a device of the user to initiate a dialogue with the online service about an activity recommended to the user by the online service from the N activities;
identify a first file in the system corresponding to the activity from N files based on the input, wherein the N files respectively correspond to the N activities;
include references in the first file to a plurality of portions of a second file in the system to conduct the dialogue, wherein the second file includes M portions for conducting dialogues about the N activities, where M is less than N, and wherein the plurality of portions are selected from the M portions based on the activity;
identify a third file in the system corresponding to a task for performing the activity, wherein the third file represents data for presenting to the user in the dialogue about the activity;
compile, at the system, the first file, the plurality of portions of the second file, and the third file to generate a handler to handle the dialogue about the activity;
receive, at the system, additional inputs from the user via the device of the user; and
conduct the dialogue with the user on the device of the user based on the additional inputs using the handler to further enhance mental health of the user.

2. The system of claim 1 wherein the instructions further configure the processor to conduct any number of dialogues with any number of users about any of the N activities using the N files, the second file, and at least N of the third file, wherein each of the N third files corresponds to a task for performing the N activities, respectively.

3. The system of claim 1 wherein the instructions further configure the processor to reuse at least one of the plurality of portions of the second file to conduct a second dialogue about a second one of the N activities with a second user of the online service.

4. The system of claim 1 wherein the instructions further configure the processor to reuse a plurality of the M portions of the second file to conduct more than one dialogue about more than one of the N activities with more than one user of the online service.

5. The system of claim 1 wherein the instructions further configure the processor to:
include a variable with a generic value in one of the plurality of portions of the second file; and
allow the first file to assign a specific value from the third file to the variable.

6. The system of claim 1 wherein the instructions further configure the processor to:
include a variable with a first value in one of the plurality of portions of the second file; and
allow the first file to overwrite the first value with a second value from the third file.

7. The system of claim 1 wherein the instructions further configure the processor to:
include a variable with a default value in one of the plurality of portions of the second file; and
allow the default value to persist in the dialogue by entering a null value for the variable in the third file.

8. The system of claim 1 wherein the instructions further configure the processor to:
conduct the dialogue based on a flow of the plurality of portions of the second file; and
control the flow in an order that is different than that in which the plurality of portions are arranged in the second file.

9. A system for conducting dialogues with users of an online service recommending N activities for enhancing mental health of the users, where N is an integer greater than 1, the system comprising:
a processor; and
memory storing instructions which when executed by the processor configure the processor to:
generate a first file including M portions for conducting the dialogues with the users of the online service about the N activities, where M is less than N;
generate N second files for the N activities, respectively;
include in each of the N second files references to a plurality of the M portions of the first file;
generate a plurality of third files, wherein each of the third files corresponds to a task for performing one of the N activities; and
conduct a dialogue with one of the users of the online service about one of the N activities using one of the N second files corresponding to the one of the N activities, a plurality of the M portions of the first file referenced by the one of the N second files, and one of the third files corresponding to a task for performing the one of the N activities;
wherein the one of the N activities is associated with mental health of the user; and
wherein the dialogue enhances the mental health of the user.

10. The system of claim 9 wherein the instructions further configure the processor to conduct any of the dialogues with any of the users about any of the N activities using the first file, the N second files, and the plurality of third files.

11. The system of claim 9 wherein the instructions further configure the processor to reuse at least one of the plurality of the M portions of the first file to conduct a second dialogue about a second one of the N activities with a second one of the users of the online service.

12. The system of claim 9 wherein the instructions further configure the processor to reuse one or more of the M portions of the first file to conduct more than one dialogue about more than one of the N activities with more than one user of the online service.

13. The system of claim 9 wherein the instructions further configure the processor to:
compile, the one of the N second files corresponding to the one of the N activities, the plurality of the M portions of the first file referenced by the one of the N second files, and the one of the third files corresponding to the task for performing the one of the N activities, to generate a handler; and
conduct the dialogue using the handler.

14. The system of claim 9 wherein the instructions further configure the processor to:
include variables with assignable values in some of the M portions of the first file;
include data in the third files for presenting to the users in the dialogues; and
allow some of the N second files to assign a portion of the data from the third files to a portion of the assignable values of the variables when conducting the dialogues.

15. The system of claim 9 wherein the instructions further configure the processor to:
include variables with generic values in the M portions of the first file;
include data in the third files for presenting to the users in the dialogues; and
allow some of the N second files to assign specific values from a portion of the data in the third files to a portion of the variables when conducting the dialogues.

16. The system of claim 9 wherein the instructions further configure the processor to:
include a variable with a first value in one of the M portions of the first file;
include data in the third files for presenting to the users in the dialogues; and
allow one of the N second files to overwrite the first value with a second value from one of the third files.

17. The system of claim 9 wherein the instructions further configure the processor to:
include a variable with a default value in one of the M portions of the first file;
include data in the third files for presenting to the users in the dialogues; and
allow the default value to persist in one of the dialogues by entering a null value for the variable in one of the third files.

18. The system of claim 9 wherein the instructions further configure the processor to:
conduct the dialogue based on a flow of the plurality of the M portions of the first file; and
control the flow in an order that is different than that in which the plurality of the M portions of the first file are arranged in the first file.

19. The system of claim 9 wherein the instructions further configure the processor to:
receive an input from the one of the users via a device of the one of the users to initiate the dialogue;
identify the one of the N second files based on the input;
receive additional inputs from the user via the device of the user; and
conduct the dialogue on the device of the user based on the additional inputs.
